(19) United States
(12) Patent Application Publication
(10) Pub. No.: US 2026/0007302 A1
(43) Pub. Date: Jan. 8, 2026

(54) MEDICAL SUPPORT DEVICE, ENDOSCOPE SYSTEM, MEDICAL SUPPORT METHOD, AND PROGRAM

(71) Applicant: FUJIFILM Corporation, Tokyo (JP)

(72) Inventor: Seiya TAKENOUCHI, Kanagawa (JP)

(73) Assignee: FUJIFILM Corporation, Tokyo (JP)

(21) Appl. No.: 19/324,171

(22) Filed: Sep. 10, 2025

Related U.S. Application Data
(63) Continuation of application No. PCT/JP2024/006789, filed on Feb. 26, 2024.

(30) Foreign Application Priority Data
Mar. 28, 2023 (JP) 2023-052125

(51) Int. Cl. A61B 1/00; A61B 1/31; G06T 7/50; G06T 7/60; G06T 7/70; G06V 10/25; G06V 10/764

(52) U.S. Cl. CPC A61B 1/000094; A61B 1/000096; A61B 1/0005; G06T 7/50; G06T 7/60; G06T 7/70; G06V 10/25; G06V 10/764; A61B 1/31; G06T 2207/10068; G06T 2207/30096; G06V 2201/03

(57) ABSTRACT

A medical support device includes a processor. The processor is configured to acquire a characteristic of each of a plurality of observation target regions, the plurality of observation target regions being recognized by performing a recognition process on a medical image in which the plurality of observation target regions appear. The processor is configured to display the medical image in a first display region. The processor is configured to display, in accordance with the characteristic, a plurality of extracted images in a second display region outside the first display region, the plurality of extracted images being images in which the plurality of observation target regions have been individually extracted from the medical image.

### CROSS-REFERENCE TO RELATED APPLICATIONS

This application is a continuation application of International Application No. PCT/JP2024/006789, filed Feb. 26, 2024, the disclosure of which is incorporated herein by reference in its entirety. Further, this application claims priority from Japanese Patent Application No. 2023-052125, filed Mar. 28, 2023, the disclosure of which is incorporated herein by reference in its entirety.

### BACKGROUND

### 1. Technical Field

The technology of the present disclosure relates to a medical support device, an endoscope system, a medical support method, and a program.

### 2. Related Art

WO2020/110214A discloses an endoscope system including an image input unit, a lesion detection unit, an oversight risk analysis unit, a notification control unit, and a notification unit.

In the endoscope system described in WO2020/110214A, a plurality of observation images obtained by imaging a photographic subject with an endoscope are sequentially input to the image input unit. The lesion detection unit detects a lesion area to be observed with the endoscope from the observation images. The oversight risk analysis unit determines a degree of oversight risk, which is a risk of an operator overlooking the lesion area, based on the observation images. The notification control unit controls a notification means and a notification method of detection of the lesion area, based on the degree of oversight risk. The notification unit notifies the operator of the detection of the lesion area under the control of the notification control unit.

In the endoscope system described in WO2020/110214A, the oversight risk analysis unit includes a lesion analysis unit that analyzes the oversight risk based on the state of the lesion area. The lesion analysis unit includes a lesion size analysis unit that estimates the size of the lesion area itself.

### SUMMARY

An embodiment according to the technology of the present disclosure provides a medical support device, an endoscope system, a medical support method, and a program that enable a user or the like to visually recognize a plurality of observation target regions appearing in a medical image in a manner that enables the user or the like to grasp the characteristics of each of the plurality of observation target regions, without impairing the visibility of the medical image.

A first aspect according to the technology of the present disclosure is a medical support device including a processor, the processor being configured to acquire a characteristic of each of a plurality of observation target regions, the plurality of observation target regions being recognized by performing a recognition process on a medical image in which the plurality of observation target regions appear; display the medical image in a first display region; and display a plurality of extracted images in a second display region outside the first display region in accordance with the characteristic, each of the plurality of extracted images being obtained by extracting a corresponding one of the plurality of observation target regions from the medical image.

A second aspect according to the technology of the present disclosure is the medical support device according to the first aspect, in which the characteristic includes a size.

A third aspect according to the technology of the present disclosure is the medical support device according to the second aspect, in which the second display region displays the plurality of extracted images in a display style that enables visual identification of a relation ship in the size between the plurality of observation target regions.

A fourth aspect according to the technology of the present disclosure is the medical support device according to the second aspect or the third aspect, in which the size is classified into a plurality of first ranges, and the second display region displays the plurality of extracted images in a manner in which the plurality of extracted images are grouped for each of the first ranges.

A fifth aspect according to the technology of the present disclosure is the medical support device according to the fourth aspect, in which the second display region displays an extracted image representative of each of the first ranges among the extracted images in a case in which the plurality of extracted images are grouped for each of the first ranges, and displays information related to the number of extracted images grouped in each of the first ranges.

A sixth aspect according to the technology of the present disclosure is the medical support device according to the fourth aspect or the fifth aspect, in which the second display region displays the plurality of extracted images in a manner in which the plurality of extracted images are grouped for each of the first ranges in a case in which the number of the plurality of extracted images exceeds a predetermined number.

A seventh aspect according to the technology of the present disclosure is the medical support device according to any one of the first to sixth aspects, in which the characteristic includes a depth.

An eighth aspect according to the technology of the present disclosure is the medical support device according to the seventh aspect, in which the second display region displays the plurality of extracted images in a display style that enables visual identification of a relationship in the depth between the plurality of observation target regions.

A ninth aspect according to the technology of the present disclosure is the medical support device according to the seventh aspect or the eighth aspect, in which the depth is classified into a plurality of second ranges, and the second display region displays the plurality of extracted images in a manner in which the plurality of extracted images are grouped for each of the second ranges.

A tenth aspect according to the technology of the present disclosure is the medical support device according to the ninth aspect, in which the second display region displays an extracted image representative of each of the second ranges among the extracted images in a case in which the plurality of extracted images are grouped for each of the second ranges, and displays information related to the number of extracted images grouped in each of the second ranges.

An eleventh aspect according to the technology of the present disclosure is the medical support device according to the ninth aspect or the tenth aspect, in which the second display region displays the plurality of extracted images in a manner in which the plurality of extracted images are grouped for each of the second ranges in a case in which the number of the plurality of extracted images exceeds a predetermined number.

A twelfth aspect according to the technology of the present disclosure is the medical support device according to any one of the first to eleventh aspects, in which the processor is configured to display positional relationship identification information on a screen, and the positional relationship identification information is information enabling identification of a correspondence relationship between a first display position at which at least one extracted image among the plurality of extracted images is displayed and a second display position at which an observation target region appearing in the at least one extracted image displayed at the first display position among the observation target regions is displayed in the first display region.

A thirteenth aspect according to the technology of the present disclosure is the medical support device according to any one of the first to twelfth aspects, in which the second display region displays the plurality of extracted images in a manner in which the plurality of extracted images are grouped for each common characteristic.

A fourteenth aspect according to the technology of the present disclosure is the medical support device according to the thirteenth aspect, in which the second display region displays, for each common characteristic, an extracted image representative of the characteristic among the extracted images in a case in which the plurality of extracted images are grouped for each common characteristic, and displays information related to the number of extracted images grouped in the common characteristic.

A fifteenth aspect according to the technology of the present disclosure is the medical support device according to the thirteenth aspect or the fourteenth aspect, in which the second display region displays the plurality of extracted images in a manner in which the plurality of extracted images are grouped for each common characteristic in a case in which the number of the plurality of extracted images exceeds a predetermined number.

A sixteenth aspect according to the technology of the present disclosure is the medical support device according to any one of the first to fifteenth aspects, in which the processor is configured to display, in a third display region, position identification information enabling identification of display positions, in the medical image, of the observation target regions included in the extracted images.

A seventeenth aspect according to the technology of the present disclosure is the medical support device according to the sixteenth aspect, in which the position identification information is a map enabling identification of the display positions in the medical image.

An eighteenth aspect according to the technology of the present disclosure is the medical support device according to the seventeenth aspect, in which the recognition process is an object recognition process using machine learning, and the map is generated based on a probability map obtained by performing the object recognition process.

A nineteenth aspect according to the technology of the present disclosure is the medical support device according to any one of the sixteenth to eighteenth aspects, in which the third display region is located at a different position from the first display region and the second display region.

A twentieth aspect according to the technology of the present disclosure is the medical support device according to any one of the first to nineteenth aspects, in which respective actual sizes of the plurality of observation target regions are measured, and the second display region displays the actual sizes, each corresponding to a corresponding one of the plurality of extracted images, in a manner that enables identification of a correspondence relationship with the plurality of extracted images.

A twenty-first aspect according to the technology of the present disclosure is the medical support device according to any one of the first to twentieth aspects, in which the extracted images are images extracted from the medical image using frames that make size differences of the observation target regions visually distinguishable between the plurality of extracted images in a case in which the plurality of extracted images are compared.

A twenty-second aspect according to the technology of the present disclosure is the medical support device according to the twenty-first aspect, in which the frames have a shape and a size that are common to the plurality of observation target regions.

A twenty-third aspect according to the technology of the present disclosure is the medical support device according to any one of the first to twenty-second aspects, in which the medical image is an endoscopic image obtained by imaging with an endoscope.

A twenty-fourth aspect according to the technology of the present disclosure is the medical support device according to any one of the first to twenty-third aspects, in which the observation target regions are lesions.

A twenty-fifth aspect according to the technology of the present disclosure is an endoscope system including the medical support device according to any one of the first to twenty-fourth aspects, and an endoscope to be inserted into a body to acquire the medical image by imaging an inside of the body.

A twenty-sixth aspect according to the technology of the present disclosure is a medical support method including acquiring a characteristic of each of a plurality of observation target regions, the plurality of observation target regions being recognized by performing a recognition process on a medical image in which the plurality of observation target regions appear; displaying the medical image in a first display region; and displaying a plurality of extracted images in a second display region outside the first display region in accordance with the characteristic, each of the plurality of extracted images being obtained by extracting a corresponding one of the plurality of observation target regions from the medical image.

A twenty-seventh aspect according to the technology of the present disclosure is the medical support method according to the twenty-sixth aspect, including using an endoscope to be inserted into a body to acquire the medical image by imaging an inside of the body.

A twenty-eighth aspect according to the technology of the present disclosure is a program for causing a computer to execute a medical support process, the medical support process including acquiring a characteristic of each of a plurality of observation target regions, the plurality of observation target regions being recognized by performing a recognition process on a medical image in which the plurality of observation target regions appear; displaying the medical image in a first display region; and displaying a plurality of extracted images in a second display region outside the first display region in accordance with the characteristic, each of the plurality of extracted images being obtained by extracting a corresponding one of the plurality of observation target regions from the medical image.

### BRIEF DESCRIPTION OF THE DRAWINGS

Exemplary embodiments according to the technique of the present disclosure will be described in detail based on the following figures, wherein:

FIG. 1 is a conceptual diagram illustrating an example of an aspect in which an endoscope system is used;

FIG. 2 is a conceptual diagram illustrating an example overall configuration of the endoscope system;

FIG. 3 is a block diagram illustrating an example hardware configuration of an electric system of the endoscope system;

FIG. 4 is a block diagram illustrating an example of functions of main components, according to an embodiment, of a processor included in a medical support device, and an example of information stored in an NVM;

FIG. 5 is a conceptual diagram illustrating an example of the content of a process of a recognition unit and a control unit;

FIG. 6 is a conceptual diagram illustrating an example of the content of a process in which the recognition unit associates each of a plurality of segmentation images with a unique identifier;

FIG. 7 is a conceptual diagram illustrating an example of the content of a process in which the control unit sets a second rectangular frame for each of a plurality of lesions appearing in a frame and assigns an identifier to each of the plurality of lesions;

FIG. 8 is a conceptual diagram illustrating an example of the content of a process in which the control unit generates first information and stores the first information in a RAM;

FIG. 9 is a conceptual diagram illustrating an example of the content of a process in which an acquisition unit measures the size of a lesion;

FIG. 10 is a conceptual diagram illustrating an example of the content of a process in which the acquisition unit generates second information and stores the second information in the RAM;

FIG. 11 is a conceptual diagram illustrating an example of the content of a process in which the control unit displays a frame in a first display region and displays a plurality of local images in a second display region in accordance with the sizes of lesions;

FIG. 12A is a flowchart illustrating an example of the flow of a medical support process;

FIG. 12B is a continuation of the flowchart illustrated in FIG. 12A;

FIG. 13 is a conceptual diagram illustrating an example of the content of a process in which the acquisition unit generates third information and stores the third information in the RAM;

FIG. 14 is a conceptual diagram illustrating an example of the content of a process in which the control unit displays a frame in the first display region and displays a plurality of local images in the second display region in accordance with distance information;

FIG. 15 is a conceptual diagram illustrating an example of an aspect in which the content displayed on a screen illustrated in FIG. 11 and the content displayed on a screen illustrated in FIG. 14 are switched in accordance with a given instruction;

FIG. 16 is a conceptual diagram illustrating an example of the content of a process in which the control unit groups a plurality of local images by size range and displays the grouped local images in the second display region;

FIG. 17 is a conceptual diagram illustrating an example of the content of a process in which the control unit groups a plurality of local images by distance range and displays the grouped local images in the second display region;

FIG. 18 is a conceptual diagram illustrating an example of the content of a process in which the control unit displays a map in a third display region;

FIG. 19 is a conceptual diagram illustrating examples of a destination to which various kinds of information are to be output; and

FIG. 20 is a conceptual diagram illustrating an example of a series of processing operations in which a processor of the endoscope system provides a process execution request to an external device via a network, the external device executes a process in response to the process execution request, and the processor of the endoscope system receives a process result from the external device.

### DETAILED DESCRIPTION

An example of an embodiment of a medical support device, an endoscope system, a medical support method, and a program according to the technology of the present disclosure will be described hereinafter with reference to the accompanying drawings.

First, terms used in the following description will be described. CPU is an abbreviation for “Central Processing Unit”. GPU is an abbreviation for

“Graphics Processing Unit”. RAM is an abbreviation for “Random Access Memory”. NVM is an abbreviation for “Non-volatile memory”. EEPROM is an abbreviation for “Electrically Erasable Programmable Read-Only Memory”. ASIC is an abbreviation for “Application Specific Integrated Circuit”. PLD is an abbreviation for “Programmable Logic Device”. FPGA is an abbreviation for “Field-Programmable Gate Array”. SoC is an abbreviation for “System-on-a-chip”. SSD is an abbreviation for “Solid State Drive”. USB is an abbreviation for “Universal Serial Bus”. HDD is an abbreviation for “Hard Disk Drive”. EL is an abbreviation for “Electro-Luminescence”. CMOS is an abbreviation for “Complementary Metal Oxide Semiconductor”. CCD is an abbreviation for “Charge Coupled Device”. AI is an abbreviation for “Artificial Intelligence”. BLI is an abbreviation for “Blue Light Imaging”. LCI is an abbreviation for “Linked Color Imaging”. I/F is an abbreviation for “Interface”. SSL is an abbreviation for “Sessile Serrated Lesion”. LAN is an abbreviation for “Local Area Network”. WAN is an abbreviation for “Wide Area Network”.

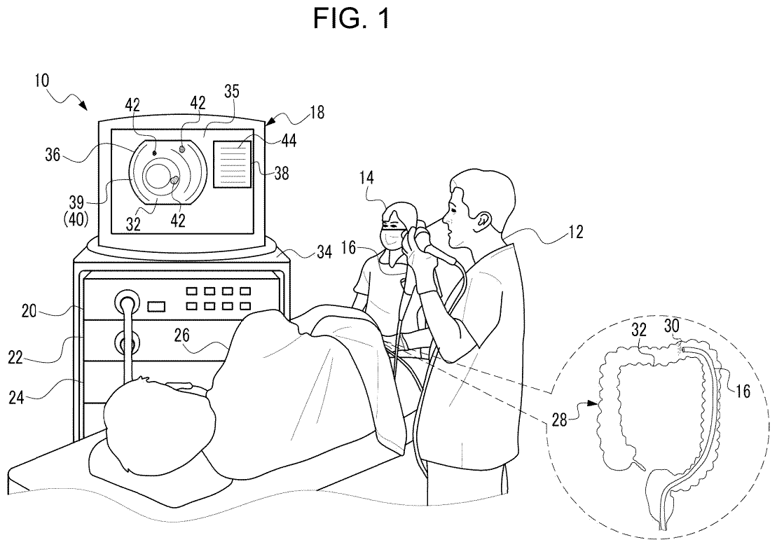

As an example, as illustrated in FIG. 1, an endoscope system 10 is used by a doctor 12 in an endoscopic examination or the like. The endoscopic examination is assisted by staff such as a nurse 14. In the present embodiment, the endoscope system 10 is an example of the “endoscope system” according to the technology of the present disclosure.

The endoscope system 10 is connected to a communication device (not illustrated) in a communicable manner, and information obtained by the endoscope system 10 is transmitted to the communication device. An example of the communication device is a server and/or a client terminal (for example, a personal computer, a tablet terminal, and/or the like) that manages various kinds of information such as electronic medical records. The communication device receives the information transmitted from the endoscope system 10 and executes a process using the received information (for example, a process of storing the information in an electronic medical record or the like).

The endoscope system 10 includes an endoscope 16, a display device 18, a light source device 20, a control device 22, and a medical support device 24. In the present embodiment, the endoscope 16 is an example of the “endoscope” according to the technology of the present disclosure.

The endoscope system 10 is a modality for performing medical care for a large intestine 28 included in the body of a subject 26 (for example, a patient) using the endoscope 16. In the present embodiment, the large intestine 28 is a target to be observed by the doctor 12.

The endoscope 16 is used by the doctor 12 and is inserted into a body cavity of the subject 26. In the present embodiment, the endoscope 16 is inserted into the large intestine 28 of the subject 26. The endoscope system 10 causes the endoscope 16 inserted into the large intestine 28 of the subject 26 to perform imaging of the inside of the large intestine 28 of the subject 26, and performs various medical treatments on the large intestine 28 as necessary.

The endoscope system 10 performs imaging of the inside of the large intestine 28 of the subject 26 to acquire an image indicating the state of the inside of the large intestine 28, and outputs the acquired image. In the present embodiment, the endoscope system 10 is an endoscope having an optical imaging function of capturing an image of reflected light obtained by irradiating the inside of the large intestine 28 with light 30 and reflecting the light 30 from an intestinal wall 32 of the large intestine 28.

While the endoscopic examination of the large intestine 28 is exemplified here, this is merely an example, and the technology of the present disclosure is also applicable to an endoscopic examination of a luminal organ such as the esophagus, the stomach, the duodenum, or the trachea.

The light source device 20, the control device 22, and the medical support device 24 are installed in a cart 34. The cart 34 is provided with a plurality of shelves along the vertical direction, and the medical support device 24, the control device 22, and the light source device 20 are installed on the shelves from bottom to top. The display device 18 is installed on top of the cart 34.

The control device 22 performs overall control of the endoscope system 10. Under the control of the control device 22, the medical support device 24 performs various kinds of image processing on an image obtained by imaging the intestinal wall 32 with the endoscope 16.

The display device 18 displays various kinds of information including images. An example of the display device 18 is a liquid crystal display or an EL display. A tablet terminal with a display may be used instead of or together with the display device 18.

The display device 18 displays a screen 35. The screen 35 includes a plurality of display regions. The plurality of display regions are arranged side by side on the screen 35. In the example illustrated in FIG. 1, a first display region 36 and a second display region 38 are depicted as an example of the plurality of display regions. The size of the first display region 36 is larger than the size of the second display region 38. The first display region 36 is used as a main display region, and the second display region 38 is used as a sub-display region. The relationship in size between the first display region 36 and the second display region 38 is not limited to this, and the first display region 36 and the second display region 38 may have any relationship in size so as to fit in the screen 35.

In the present embodiment, the screen 35 is an example of the “screen” according to the technology of the present disclosure, the first display region 36 is an example of the “first display region” according to the technology of the present disclosure, and the second display region 38 is an example of the “second display region” according to the technology of the present disclosure.

The first display region 36 displays an endoscopic moving image 39. The endoscopic moving image 39 is a moving image acquired by imaging the intestinal wall 32 with the endoscope 16 in the large intestine 28 of the subject 26. In the example illustrated in FIG. 1, a moving image in which the intestinal wall 32 appears is depicted as an example of the endoscopic moving image 39.

The intestinal wall 32 appearing in the endoscopic moving image 39 includes a plurality of lesions 42 (for example, in the example illustrated in FIG. 1, three lesions 42) as a plurality of regions of interest (that is, a plurality of observation target regions) to be gazed at by the doctor 12, and the doctor 12 can visually recognize the state of the intestinal wall 32 including the plurality of lesions 42 through the endoscopic moving image 39. In the present embodiment, the lesions 42 are an example of the “observation target regions” and the “lesions” according to the technology of the present disclosure.

There are various types of lesions 42, and the types of lesions 42 include, for example, neoplastic polyps and non-neoplastic polyps. The types of the neoplastic polyps include, for example, adenomatous polyps (for example, SSL). The types of the non-neoplastic polyps include, for example, hamartoma polyps, hyperplastic polyps, and inflammatory polyps. The types exemplified here are types considered in advance to be possible types of the lesions 42 when an endoscopic examination is performed on the large intestine 28, and the type of lesion differs depending on the organ on which the endoscopic examination is performed.

While the present embodiment exemplifies the lesions 42, this is merely an example. The region of interest (that is, the observation target region) to be gazed at by the doctor 12 may be an organ (for example, the duodenal papilla), a marked region, an artificial treatment tool (for example, an artificial clip), a treated region (for example, a region with a trace of removal of a polyp or the like), or the like.

The image displayed in the first display region 36 is one frame 40 included in a moving image configured to include a plurality of frames 40 along a time series. That is, the first display region 36 displays the plurality of frames 40 along a time series at a predetermined frame rate (for example, several tens of frames/second). In the present embodiment, the frame 40 is an example of the “medical image” and the “endoscopic image” according to the technology of the present disclosure.

An example of the moving image to be displayed in the first display region 36 is a live-view moving image. The live-view moving image is merely an example, and a moving image that is temporarily stored in a memory or the like before being displayed, like a post-view moving image, may be used. Alternatively, each frame included in a recording moving image stored in the memory or the like may be reproduced and displayed on the screen 35 (for example, the first display region 36) as the endoscopic moving image 39.

On the screen 35, the second display region 38 is present outside the first display region 36. In the example illustrated in FIG. 1, the second display region 38 is adjacent to the first display region 36 and is displayed on the right side of the screen 35 when viewed from the front. The second display region 38 may be displayed at any position different from the first display region 36, but is preferably displayed at a position that enables comparison with the endoscopic moving image 39 displayed in the first display region 36.

The second display region 38 displays medical information 44, which is information related to medical treatment. Examples of the medical information 44 include information and the like for assisting the doctor 12 in making a medical determination or the like. An example of the information and the like for assisting the doctor 12 in making a medical determination or the like is various kinds of information related to the subject 26 into which the endoscope 16 is inserted, various kinds of information obtained by performing processing using AI on the endoscopic moving image 39, and/or the like. The medical information 44 will be described in further detail below.

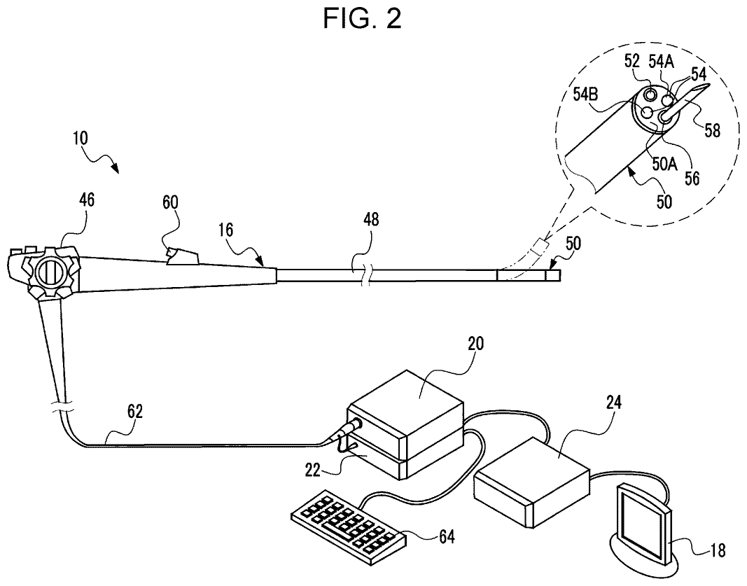

As an example, as illustrated in FIG. 2, the endoscope 16 includes an operation section 46 and an insertion section 48. The insertion section 48 partially bends in response to the operation section 46 being operated. When the doctor 12 (see FIG. 1) operates the operation section 46, the insertion section 48 is inserted into the large intestine 28 (see FIG. 1) while bending according to the shape of the large intestine 28.

The insertion section 48 has a tip portion 50 provided with a camera 52, an illumination device 54, and a treatment tool opening 56. The camera 52 and the illumination device 54 are provided on a tip surface 50A of the tip portion 50. While an example embodiment in which the camera 52 and the illumination device 54 are provided on the tip surface 50A of the tip portion 50 is given here, this is merely an example. The camera 52 and the illumination device 54 may be provided on a side surface of the tip portion 50 such that the endoscope 16 is configured as a side view endoscope.

The camera 52 is inserted into the body cavity of the subject 26 to perform imaging of an observation target region. In the present embodiment, the camera 52 performs imaging of the inside of the body of the subject 26 (for example, the inside of the large intestine 28) to acquire the endoscopic moving image 39. An example of the camera 52 is a CMOS camera. However, this is merely an example, and the camera 52 may be any other type of camera such as a CCD camera.

The illumination device 54 has illumination windows 54A and 54B. The illumination device 54 emits the light 30 (see FIG. 1) through the illumination windows 54A and 54B. Examples of the type of the light 30 to be emitted from the illumination device 54 include visible light (for example, white light or the like) and invisible light (for example, near-infrared light or the like). The illumination device 54 further emits special light through the illumination windows 54A and 54B. Examples of the special light include light for BLI and/or light for LCI. The camera 52 performs imaging of the inside of the large intestine 28 using an optical method, with the inside of the large intestine 28 irradiated with the light 30 from the illumination device 54.

The treatment tool opening 56 is an opening for allowing a treatment tool 58 to protrude from the tip portion 50. The treatment tool opening 56 is also used as a suction port for sucking blood, bodily waste, and the like, and as a delivery port for delivering a fluid.

The operation section 46 has a treatment tool insertion port 60 formed therein, and the treatment tool 58 is inserted into the insertion section 48 through the treatment tool insertion port 60. The treatment tool 58 passes through the insertion section 48 and protrudes to the outside from the treatment tool opening 56. In the example illustrated in FIG. 2, a puncture needle is illustrated as the treatment tool 58 protruding from the treatment tool opening 56. While a puncture needle is exemplified as the treatment tool 58, this is merely an example, and the treatment tool 58 may be gripping forceps, a papillotomy knife, a snare, a catheter, a guide wire, a cannula, a puncture needle with a guide sheath, and/or the like.

The endoscope 16 is connected to the light source device 20 and the control device 22 through a universal cord 62. The control device 22 is connected to the medical support device 24 and a reception device 64. The medical support device 24 is also connected to the display device 18. That is, the control device 22 is connected to the display device 18 through the medical support device 24.

Since the medical support device 24 is exemplified here as an external device for extending the functions implemented by the control device 22, an example embodiment in which the control device 22 and the display device 18 are indirectly connected through the medical support device 24 is given here, although this is merely an example. For example, the display device 18 may be directly connected to the control device 22. In this case, for example, the control device 22 may be mounted with the functions of the medical support device 24, or the control device 22 may be mounted with a function of causing a server (not illustrated) to execute the same process as a process (for example, a medical support process described below) executed by the medical support device 24 and receiving and using a process result obtained by the server.

The reception device 64 receives an instruction from the doctor 12 and outputs the received instruction to the control device 22 as an electrical signal. An example of the reception device 64 is a keyboard, a mouse, a touch panel, a foot switch, a microphone, a remote operation device, and/or the like.

The control device 22 controls the light source device 20, transmits and receives various signals to and from the camera 52, and transmits and receives various signals to and from the medical support device 24.

The light source device 20 emits light under the control of the control device 22 and supplies the light to the illumination device 54. The illumination device 54 incorporates a light guide, and the light supplied from the light source device 20 is emitted from the illumination windows 54A and 54B through the light guide. The control device 22 causes the camera 52 to perform imaging, acquires the endoscopic moving image 39 (see FIG. 1) from the camera 52, and outputs the endoscopic moving image 39 to a predetermined output destination (for example, the medical support device 24).

The medical support device 24 performs various kinds of image processing on the endoscopic moving image 39 input from the control device 22 to support medical treatment (here, as an example, endoscopy). The medical support device 24 outputs the endoscopic moving image 39 on which the various kinds of image processing have been performed to a predetermined output destination (for example, the display device 18).

While an example embodiment has been described in which the endoscopic moving image 39 output from the control device 22 is output to the display device 18 through the medical support device 24, this is merely an example. For example, in another aspect, the control device 22 and the display device 18 may be connected to each other, and the endoscopic moving image 39 on which image processing has been performed by the medical support device 24 may be displayed on the display device 18 through the control device 22.

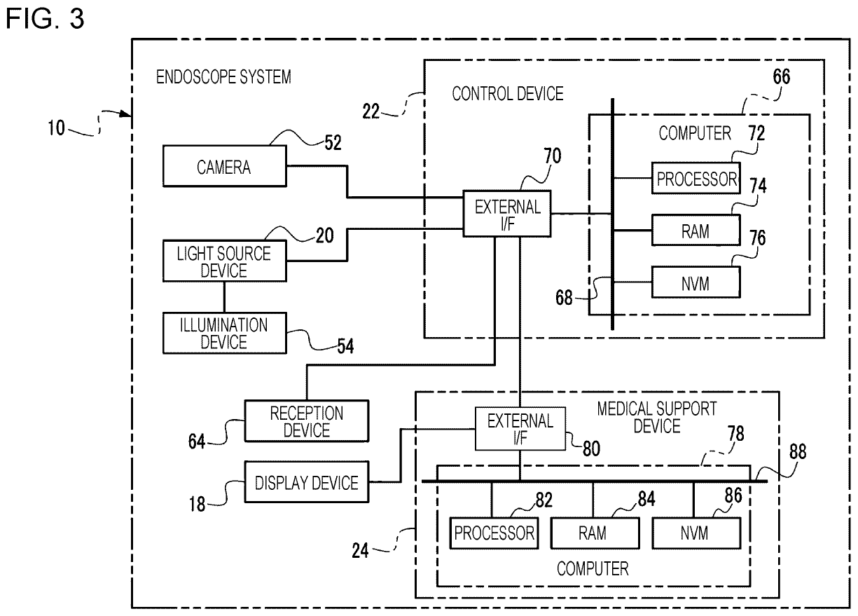

As an example, as illustrated in FIG. 3, the control device 22 includes a computer 66, a bus 68, and an external I/F 70. The computer 66 includes a processor 72, a RAM 74, and an NVM 76. The processor 72, the RAM 74, the NVM 76, and the external I/F 70 are connected to the bus 68.

For example, the processor 72 has at least one CPU and at least one GPU and controls the entire control device 22. The GPU operates under the control of the CPU and is responsible for performing various kinds of graphics-based processing, arithmetic operations using a neural network, and the like. The processor 72 may include one or more CPUs with integrated GPU functions, or may include one or more CPUs without integrated GPU functions. In the example illustrated in FIG. 3, the computer 66 is mounted with one processor 72. However, this is merely an example, and the computer 66 may be mounted with a plurality of processors 72.

The RAM 74 is a memory that temporarily stores information and is used as a work memory by the processor 72. The NVM 76 is a non-volatile storage device that stores various programs, various parameters, and the like. An example of the NVM 76 is a flash memory (for example, an EEPROM and/or an SSD). The flash memory is merely an example, and the NVM 76 may be any other non-volatile storage device such as an HDD, or a combination of two or more types of non-volatile storage devices.

The external I/F 70 handles transmission and reception of various kinds of information between the processor 72 and one or more devices (hereinafter also referred to as “first external devices”) external to the control device 22. An example of the external I/F 70 is a USB interface.

The camera 52 is connected to the external I/F 70 as one of the first external devices, and the external I/F 70 handles transmission and reception of various kinds of information between the camera 52 and the processor 72. The processor 72 controls the camera 52 through the external I/F 70. Further, the processor 72 acquires the endoscopic moving image 39 (see FIG. 1), which is obtained by imaging the inside of the large intestine 28 (see FIG. 1) using the camera 52, through the external I/F 70.

The light source device 20 is connected to the external I/F 70 as one of the first external devices, and the external I/F 70 handles transmission and reception of various kinds of information between the light source device 20 and the processor 72. The light source device 20 supplies light to the illumination device 54 under the control of the processor 72. The illumination device 54 emits the light supplied from the light source device 20.

The reception device 64 is connected to the external I/F 70 as one of the first external devices, and the processor 72 acquires an instruction received by the reception device 64 through the external I/F 70 and executes a process corresponding to the acquired instruction.

The medical support device 24 includes a computer 78 and an external I/F 80. The computer 78 includes a processor 82, a RAM 84, and an NVM 86. The processor 82, the RAM 84, the NVM 86, and the external I/F 80 are connected to a bus 88. In the present embodiment, the medical support device 24 is an example of the “medical support device” according to the technology of the present disclosure, the computer 78 is an example of the “computer” according to the technology of the present disclosure, and the processor 82 is an example of the “processor” according to the technology of the present disclosure.

Since the hardware configuration (that is, the processor 82, the RAM 84, and the NVM 86) of the computer 78 is basically the same as the hardware configuration of the computer 66, the description of the hardware configuration of the computer 78 will be omitted here.

The external I/F 80 handles transmission and reception of various kinds of information between the processor 82 and one or more devices (hereinafter also referred to as “second external devices”) external to the medical support device 24. An example of the external I/F 80 is a USB interface.

The control device 22 is connected to the external I/F 80 as one of the second external devices. In the example illustrated in FIG. 3, the external I/F 70 of the control device 22 is connected to the external I/F 80. The external I/F 80 handles transmission and reception of various kinds of information between the processor 82 of the medical support device 24 and the processor 72 of the control device 22. For example, the processor 82 acquires the endoscopic moving image 39 (see FIG. 1) from the processor 72 of the control device 22 through the external I/Fs 70 and 80, and performs various kinds of image processing on the acquired endoscopic moving image 39.

The display device 18 is connected to the external I/F 80 as one of the second external devices. The processor 82 controls the display device 18 through the external I/F 80 to display various kinds of information (for example, the endoscopic moving image 39 and the like on which the various kinds of image processing have been performed) on the display device 18.

In an endoscopic examination, the doctor 12 determines whether a lesion 42 appearing in the endoscopic moving image 39 requires medical treatment, while checking the endoscopic moving image 39 through the display device 18, and performs medical treatment on the lesion 42, if necessary. The size of the lesion 42 is a determination factor important for determining whether medical treatment is necessary.

The recent development of machine learning has enabled the AI-based detection and classification of a lesion 42 based on the endoscopic moving image 39. Application of this technique makes it possible to measure the size of the lesion 42 from the endoscopic moving image 39. Accurately measuring the size of a lesion 42 and presenting the measurement result to the doctor 12 is very useful for the doctor 12 to perform medical treatment on the lesion 42.

When the size of a lesion 42 is measured, the measured size needs to be correctly notified to the doctor 12 to an extent that does not interfere with the endoscopic examination.

In particular, when a plurality of lesions 42 appear in the frame 40, it is required for the doctor 12 to visually recognize the plurality of lesions 42 appearing in the frame 40 in a manner that enables the doctor 12 to grasp the characteristics such as the size of each of the plurality of lesions 42, without impairing the visibility of the frame 40 displayed on the screen 35.

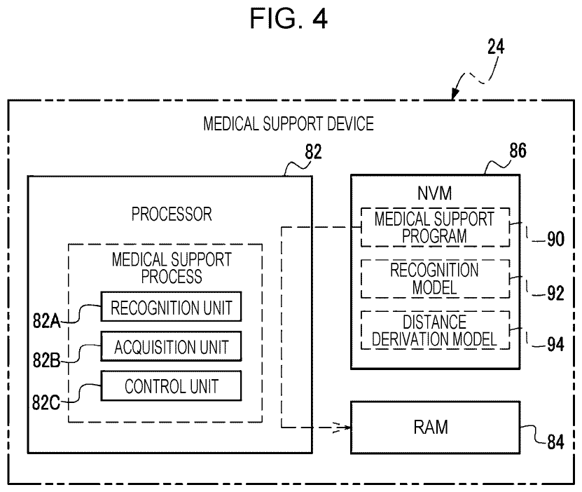

In view of such circumstances, in the present embodiment, as an example, as illustrated in FIG. 4, the processor 82 of the medical support device 24 performs a medical support process.

The NVM 86 stores a medical support program 90. The medical support program 90 is an example of the “program” according to the technology of the present disclosure. The processor 82 reads the medical support program 90 from the NVM 86 and executes the read medical support program 90 on the RAM 84 to perform the medical support process. The medical support process is implemented by the processor 82 operating as a recognition unit 82A, an acquisition unit 82B, and a control unit 82C in accordance with the medical support program 90 executed on the RAM 84.

The NVM 86 stores a recognition model 92 and a distance derivation model 94. As described in detail below, the recognition model 92 is used by the recognition unit 82A, and the distance derivation model 94 is used by the acquisition unit 82B.

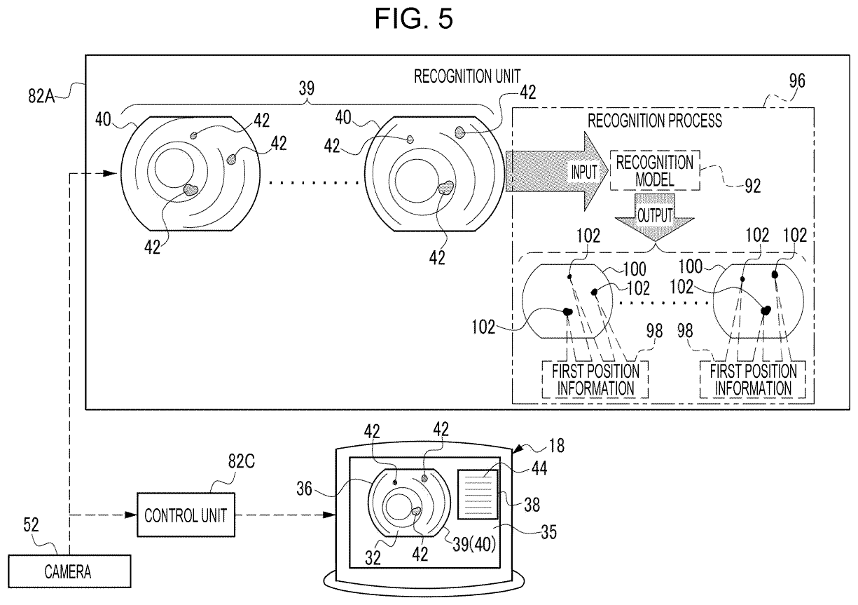

As an example, as illustrated in FIG. 5, the recognition unit 82A and the control unit 82C acquire each of a plurality of frames 40 along a time series included in the endoscopic moving image 39 generated by imaging with the camera 52 in accordance with an imaging frame rate (for example, several tens of frames/second) from the camera 52 frame by frame along a time series.

The control unit 82C outputs the endoscopic moving image 39 to the display device 18. For example, the control unit 82C displays the endoscopic moving image 39 in the first display region 36 as a live view image. That is, each time the control unit 82C acquires a frame 40 from the camera 52, the control unit 82C sequentially displays the acquired frame 40 in the first display region 36 in accordance with a display frame rate (for example, several tens of frames/second). The control unit 82C further displays the medical information 44 in the second display region 38. Further, for example, the control unit 82C updates the content (for example, the medical information 44) displayed in the second display region 38 in accordance with the content displayed in the first display region 36.

The recognition unit 82A uses the endoscopic moving image 39 acquired from the camera 52 to recognize the plurality of lesions 42 appearing in the endoscopic moving image 39. That is, the recognition unit 82A sequentially performs a recognition process 96 on each of the plurality of frames 40 along a time series included in the endoscopic moving image 39 acquired from the camera 52 to recognize the plurality of lesions 42 appearing in each of the frames 40. For example, the recognition unit 82A recognizes the geometric characteristics (for example, the position, the shape, and the like) of each of the plurality of lesions 42, the type of each of the plurality of lesions 42, the category of each of the plurality of lesions 42 (for example, pedunculated, sub-pedunculated, sessile, superficial elevated, superficial flat, superficial depressed, and the like), and the like.

Each time a frame 40 is acquired, the recognition unit 82A performs the recognition process 96 on the acquired frame 40. The recognition process 96 is a process for recognizing the plurality of lesions 42 by a method using AI (that is, an object recognition process using machine learning). In the present embodiment, for example, an AI-based object recognition process using a segmentation method (for example, semantic segmentation, instance segmentation, and/or panoptic segmentation) is used as the recognition process 96.

A process using the recognition model 92 is performed as the recognition process 96. The recognition model 92 is a trained model for object recognition using an AI-based segmentation method. An example of the trained model for object recognition using an AI-based segmentation method is a model for semantic segmentation. An example of the model for semantic segmentation is an encoder-decoder structure model. An example of the encoder-decoder structure model is a U-Net model, an HRNet model, or the like. In the present embodiment, the recognition process 96 is an example of the “recognition process” and the “object recognition process” according to the technology of the present disclosure.

The recognition model 92 is optimized by training a neural network through machine learning using first training data. The first training data is a dataset including a plurality of pieces of data (that is, data for a plurality of frames) in which first example data and first ground-truth data are associated with each other.

The first example data is an image corresponding to the frame 40. The first ground-truth data is ground-truth data (that is, an annotation) for the first example data. An example of the first ground-truth data is an annotation for identifying the geometric characteristics, type, and category of a lesion appearing in an image used as the first example data.

The recognition unit 82A acquires a frame 40 from the camera 52 and inputs the acquired frame 40 to the recognition model 92. Accordingly, each time a frame 40 is input, the recognition model 92 identifies the geometric characteristics of each of the plurality of lesions 42 appearing in the input frame 40 and outputs information enabling the identification of the geometric characteristics of each of the plurality of lesions 42. In the example illustrated in FIG. 5, a probability map 100, which is information enabling the identification of the positions of the lesions 42 in the frame 40, is depicted as an example of the information enabling the identification of the geometric characteristics. Further, the recognition unit 82A acquires, from the recognition model 92, information indicating the type of each of the plurality of lesions 42 appearing in the frame 40 input to the recognition model 92, and information indicating the category of each of the plurality of lesions 42 appearing in the frame 40 input to the recognition model 92.

Each time a frame 40 is input to the recognition model 92, the recognition unit 82A acquires, from the recognition model 92, a probability map 100 related to the frame 40 input to the recognition model 92. The probability map 100 is a map in which the distribution of the positions of the lesions 42 in the frame 40 is expressed in terms of a probability, which is an example of a measure of the likelihood. The probability map 100 is typically referred to also as a reliability map, a certainty map, or the like.

The probability map 100 includes a plurality of segmentation images 102 that define the plurality of lesions 42 recognized by the recognition unit 82A. The segmentation images 102 are image regions for identifying the positions of the lesions 42, which are recognized by performing the recognition process 96 on the frame 40, in the frame 40 (that is, images displayed in a display style that enables the identification of the positions where the lesions 42 are most likely to be present in the frame 40). Each of the segmentation images 102 is associated with first position information 98 by the recognition unit 82A. An example of the first position information 98 in this case is coordinates that enable the identification of the position of the segmentation image 102 on the probability map 100 (in other words, coordinates that enable the identification of the display position of the lesion 42 in the frame 40). The position of the segmentation image 102 on the probability map 100 refers to, for example, the position of the outer contour of the segmentation image 102 on the probability map 100.

The probability map 100 may be displayed on the screen 35 (for example, in the second display region 38) as the medical information 44 by the control unit 82C. In this case, the probability map 100 displayed on the screen 35 is updated in accordance with the display frame rate applied to the first display region 36. That is, the display of the probability map 100 in the second display region 38 (that is, the display of the segmentation images 102) is updated in synchronization with the display timing of the endoscopic moving image 39 displayed in the first display region 36. This configuration allows the doctor 12 to grasp the schematic positions of the lesions 42 in the endoscopic moving image 39 displayed in the first display region 36 by referring to the probability map 100 displayed in the second display region 38 while observing the endoscopic moving image 39 displayed in the first display region 36.

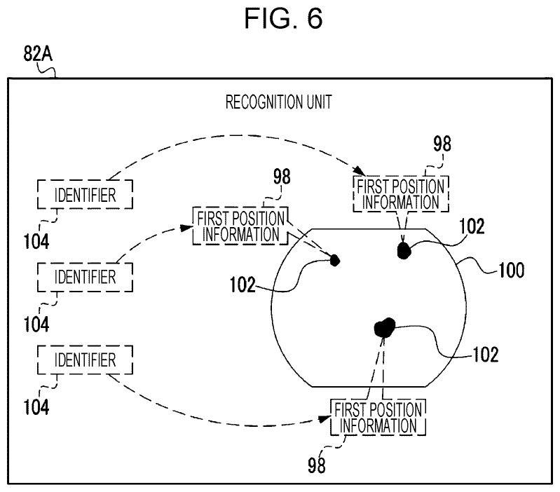

As an example, as illustrated in FIG. 6, the recognition unit 82A associates each of the plurality of segmentation images 102 on the probability map 100 with an identifier 104 that can individually identify a corresponding one of the segmentation images 102. The identifier 104 is an identifier unique to each of the segmentation images 102. Each of the identifiers 104 is associated with a corresponding the segmentation image 102 by assigning each of the identifiers 104 to a piece of first position information 98 corresponding to a corresponding the segmentation images 102. In the present embodiment, the identifier 104 is an example of the “positional relationship identification information” according to the technology of the present disclosure.

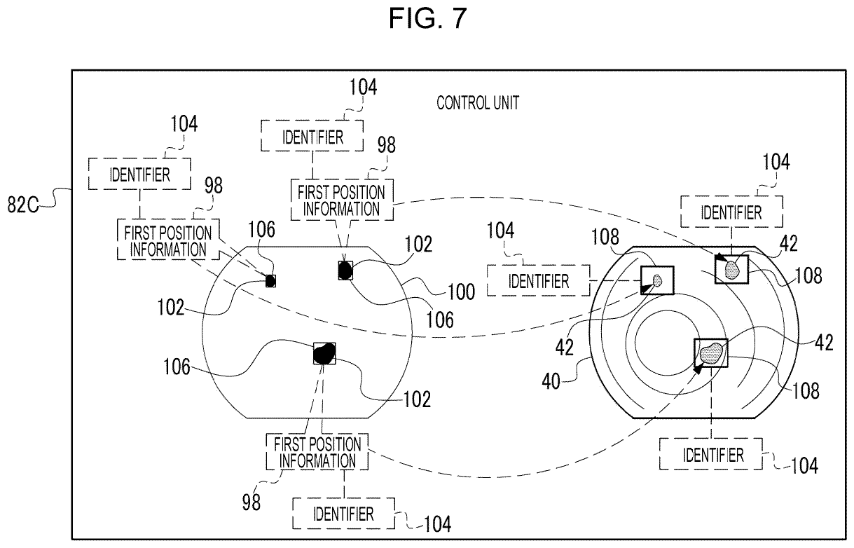

As an example, as illustrated in FIG. 7, the control unit 82C creates, for each of the plurality of segmentation images 102 on the probability map 100, a first rectangular frame 106 circumscribing the segmentation image 102, based on the corresponding piece of first position information 98. Then, the control unit 82C creates a plurality of second rectangular frames 108 in the frame 40, based on the plurality of first rectangular frames 106 created on the probability map 100. The second rectangular frame 108 is a rectangular frame which encloses the lesion 42 in the frame 40, and are each assigned to a corresponding one of the plurality of lesions 42 appearing in the frame 40. The second rectangular frame 108 is obtained by enlarging the largest first rectangular frame 106 among the plurality of first rectangular frames 106 created on the probability map 100 with a predetermined magnification (for example, a magnification larger than 1).

The control unit 82C associates the lesion 42 corresponding to the segmentation image 102 on the probability map 100 with the same identifier 104 as the identifier 104 associated with the segmentation image 102. That is, the segmentation images 102 and one of the lesions 42 having a correspondence relationship in position between the probability map 100 and the frame 40 are associated with a common identifier 104. An identifier 104 is associated with each of the lesions 42 by assigning the identifier 104 associated with the segmentation image 102 corresponding to the lesion 42 to the second rectangular frame 108 assigned to the lesion 42 corresponding to the segmentation image 102.

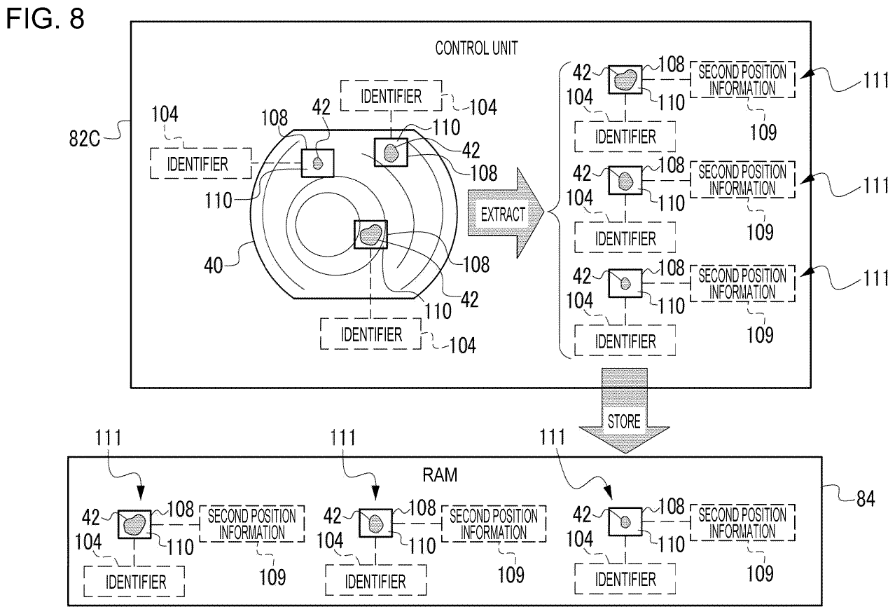

As an example, as illustrated in FIG. 8, the control unit 82C extracts a plurality of local images 110 in which different lesions 42 appear, from the frame 40 in which the lesions 42 are associated with the second rectangular frames 108 and the identifiers 104. The local images 110 are local images in the frame 40. In the example illustrated in FIG. 8, as an example of the local images 110, images enclosed by the second rectangular frames 108 in the frame 40 are illustrated. That is, the plurality of local images 110 are images obtained by individually extracting the plurality of lesions 42 appearing in the frame 40 from the frame 40 by using second rectangular frames 108 that are frames having the same shape and the same size (in other words, individually cropped images).

As described above, the plurality of local images 110 are images obtained by individually extracting the plurality of lesions 42 from the frame 40 by using second rectangular frames 108 that are frames having the same shape and the same size. Thus, when the plurality of local images 110 are compared, the size differences of the lesions 42 between the plurality of local images 110 are visually distinguishable.

In the present embodiment, the local images 110 are an example of the “extracted images” according to the technology of the present disclosure. In the present embodiment, the second rectangular frames 108 are an example of the “frames” according to the technology of the present disclosure.

The control unit 82C generates first information 111 for each of the lesions 42 appearing in the frame 40 and stores the first information 111 in the RAM 84. The first information 111 is information in which the corresponding identifier 104, the corresponding local image 110, and the corresponding second position information 109 are associated with each other. In this case, for example, the control unit 82C assigns an identifier 104 and second position information 109 to each of the local images 110 extracted from the frame 40 to generate first information 111. The identifier 104 assigned to each of the local images 110 is an identifier 104 assigned to the second rectangular frame 108 used to extract the local image 110 from the frame 40. The second position information 109 is information (for example, coordinates) enabling the identification of the position of a corresponding one of the local images 110 in the frame 40.

While an example embodiment in which a plurality of local images 110 are extracted from the frame 40 by using second rectangular frames 108 is given here, this is merely an example. A plurality of images may be extracted from the frame 40 by using, for example, frames having a different shape and size from the second rectangular frames 108. Also in this case, like the second rectangular frames 108, frames having a shape and a size common to the plurality of lesions 42 are used. That is, frames are used that make the size differences of the lesions 42 between the plurality of extracted images (in the example illustrated in FIG. 8, the plurality of local images 110) extracted from the frame 40 visually distinguishable when the plurality of extracted images are compared.

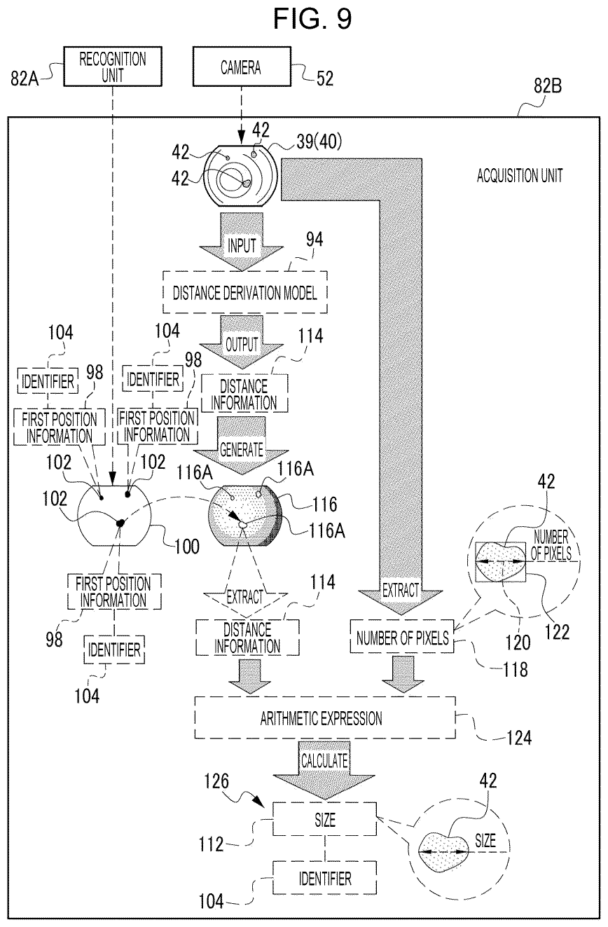

As an example, as illustrated in FIG. 9, the acquisition unit 82B acquires a frame 40 from the camera 52 and acquires a size 112 of a lesion 42 appearing in the frame 40 acquired from the camera 52 (as an example, the frame 40 used in the recognition process 96). The size 112 of the lesion 42 appearing in the frame 40 is acquired by the acquisition unit 82B measuring the size 112. The acquisition unit 82B measures the size 112, based on the frame 40. In the present embodiment, the acquisition unit 82B measures the size 112 of the lesion 42 in time series, based on each of the plurality of frames 40 included in the endoscopic moving image 39 acquired from the camera 52. The size 112 of the lesion 42 refers to the size of the lesion 42 in real space. In the following, the size of the lesion 42 in real space is also referred to as an “actual size”, for convenience of description. In the present embodiment, the size 112 is an example of the “characteristic”, the “size”, and the “actual size” according to the technology of the present disclosure.

To implement the measurement of the size 112 of the lesion 42, the acquisition unit 82B acquires distance information 114 of the lesion 42, based on the frame 40 acquired from the camera 52. The distance information 114 is information indicating the distance from the camera 52 (that is, the observation position) to the intestinal wall 32 (see FIG. 1) including the lesion 42. While the distance from the camera 52 to the intestinal wall 32 including the lesion 42 is exemplified here, this is merely an example. Instead of the distance, a numerical value indicating the depth from the camera 52 to the intestinal wall 32 including the lesion 42 (for example, a plurality of numerical values defining depths in a stepwise manner (for example, numerical values in several steps to several tens of steps)) may be used.

The acquisition unit 82B acquires the distance information 114 for the following reason. Even for lesions 42 having the same size, the farther the positions of the lesions 42 are from the camera 52, the smaller the sizes of the lesions 42 are in the image, and thus it is necessary to take into account how far the positions of the lesions 42 are from the camera 52 to determine the actual sizes.

The distance information 114 is acquired for each of all the pixels constituting the frame 40. The distance information 114 may be acquired for each block (for example, a pixel group constituted by several pixels to several hundreds of pixels), which is larger than a pixel in the frame 40.

The acquisition unit 82B acquires the distance information 114 by, for example, deriving the distance information 114 using an AI-based method. In the present embodiment, the distance derivation model 94 is used to derive the distance information 114.

The distance derivation model 94 is optimized by training the neural network through machine learning using second training data. The second training data is a dataset including a plurality of pieces of data (that is, data for a plurality of frames) in which second example data and second ground-truth data are associated with each other.

The second example data is an image corresponding to the frame 40. The second ground-truth data is ground-truth data (that is, an annotation) for the second example data. An example of the second ground-truth data is an annotation for identifying a distance corresponding to each pixel appearing in an image used as the second example data.

The acquisition unit 82B acquires a frame 40 from the camera 52 and inputs the acquired frame 40 to the distance derivation model 94. As a result, the distance derivation model 94 outputs the distance information 114 on a pixel-by-pixel basis in the frame 40 that has been input. That is, in the acquisition unit 82B, information indicating the distance from the position of the camera 52 (for example, the position of the image sensor, the objective lens, or the like mounted in the camera 52) to the intestinal wall 32 appearing in the frame 40 is output from the distance derivation model 94 as the distance information 114 on a pixel-by-pixel basis in the frame 40.

The acquisition unit 82B generates a distance image 116, based on the distance information 114 output from the distance derivation model 94. The distance image 116 is an image in which the distance information 114 is distributed in units of pixels included in the endoscopic moving image 39 (that is, the frame 40).

The acquisition unit 82B acquires the first position information 98 assigned to the segmentation images 102 on the probability map 100 obtained by the recognition unit 82A. The acquisition unit 82B refers to the first position information 98 and extracts the distance information 114 from a segmentation-corresponding region 116A in the distance image 116. The segmentation-corresponding region 116A is a region corresponding to a position identified from the first position information 98 in the distance image 116. The distance information 114 extracted from the segmentation-corresponding region 106A includes, for example, the distance information 114 corresponding to the position (for example, centroid) of the lesion 42, or a statistical measure (for example, the median, mean, or mode) based on the distance information 114 for a plurality of pixels (for example, all the pixels) included in the lesion 42.

The acquisition unit 82B extracts the number of pixels 118 from the frame 40. The number of pixels 118 is the number of pixels on a line segment 120 crossing an image region located at a position identified from the first position information 98 (that is, an image region indicating the lesion 42) within an entire image region of the frame 40 input to the distance derivation model 94. An example of the line segment 120 is the longest line segment parallel to the long sides of a rectangular frame 122 circumscribing the image region indicating the lesion 42. The line segment 120 is merely an example. Instead of the line segment 120, the longest line segment parallel to the short sides of the rectangular frame 122 circumscribing the image region indicating the lesion 42 may be used.

The acquisition unit 82B calculates the size 112 of the lesion 42, based on the distance information 114 extracted from the segmentation-corresponding region 116A in the distance image 116 and the number of pixels 118 extracted from the frame 40. The size 112 is calculated using an arithmetic expression 124. The arithmetic expression 124 is an arithmetic expression in which the distance information 114 and the number of pixels 118 are independent variables and the size 112 is a dependent variable. The acquisition unit 82B inputs the distance information 114 extracted from the distance image 116 and the number of pixels 118 extracted from the frame 40 to the arithmetic expression 124. The arithmetic expression 124 outputs the size 112 corresponding to the distance information 114 and the number of pixels 118 that have been input. The acquisition unit 82B generates second information 126. The second information 126 is generated by associating an identifier 104 with the size 112 output from the arithmetic expression 124. The identifier 104 to be associated with the size 112 is the identifier 104 associated with the segmentation image 102 corresponding to the segmentation-corresponding region 116A used to calculate the size 112.

While the length of the lesion 42 in real space is illustrated as the size 112, the technology of the present disclosure is not limited to this. The size 112 may be the surface area or volume of the lesion 42 in real space. In this case, for example, as the arithmetic expression 124, an arithmetic expression is used in which the number of pixels in an entire image region indicating the lesion 42 and the distance information 114 are independent variables and the surface area or volume of the lesion 42 in real space is a dependent variable.

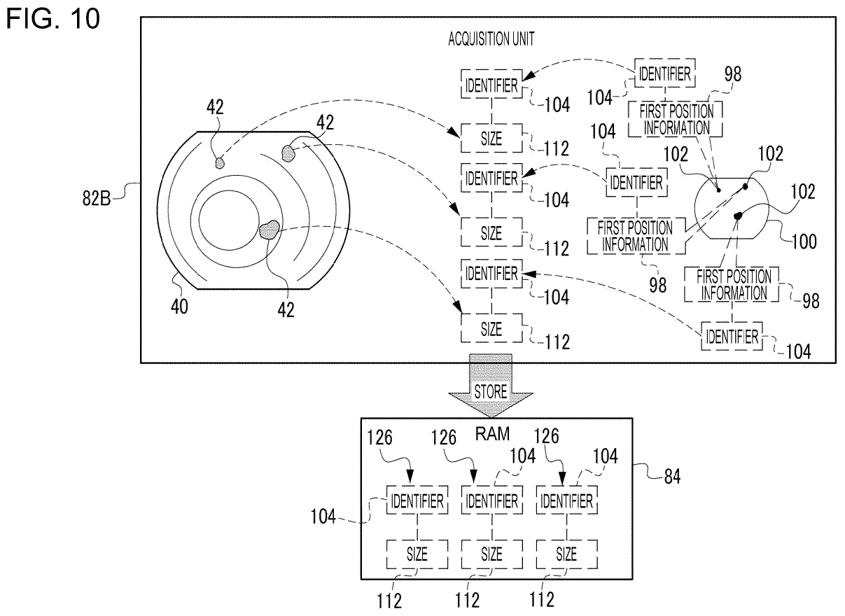

As an example, as illustrated in FIG. 10, the acquisition unit 82B also acquires the size 112 of another lesion 42 appearing in the frame 40 in a manner similar to that in the example illustrated in FIG. 9, and assigns the identifier 104 associated with the segmentation image 102 corresponding to the segmentation-corresponding region 116A used to calculate the size 112 to the acquired size 112 to generate the second information 126. Then, the acquisition unit 82B stores, in the RAM 84, the second information 126 generated for each of the plurality of lesions 42 appearing in the frame 40.

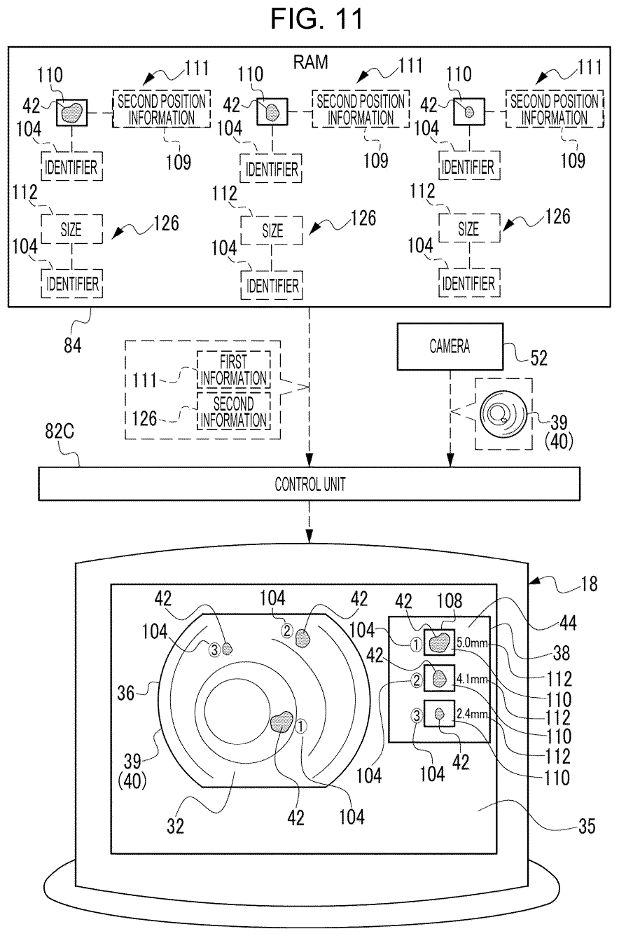

As an example, as illustrated in FIG. 11, the control unit 82C acquires the sizes 112 from the acquisition unit 82B. The control unit 82C further acquires, from the camera 52, the frame 40 used to measure the sizes 112 by the acquisition unit 82B. The control unit 82C displays the frame 40 acquired from the camera 52 in the first display region 36.

The control unit 82C further acquires, from the RAM 84, a plurality of pieces of first information 111 and a plurality of pieces of second information 126 corresponding to the plurality of lesions 42 appearing in the frame 40. The control unit 82C displays a plurality of identifiers 104 in the frame 40 displayed in the first display region 36, based on the plurality of pieces of first information 111 and the plurality of pieces of second information 126 acquired from the RAM 84. The control unit 82C further displays the plurality of identifiers 104, the plurality of local images 110, and the plurality of sizes 112 in the second display region 38 as portions of the medical information 44, based on the plurality of pieces of first information 111 and the plurality of pieces of second information 126 acquired from the RAM 84.

Each time the acquisition unit 82B acquires the size 112, the control unit 82C displays the latest identifier 104, the latest local image 110, and the latest size 112 on the screen 35. That is, the identifier 104, the local image 110, and the size 112 displayed on the screen 35 are updated to the latest identifier 104, the latest local image 110, and the latest size 112, respectively, each time the acquisition unit 82B acquires the size 112.

The plurality of identifiers 104 are displayed superimposed on the frame 40 in the first display region 36. In this case, for example, the plurality of identifiers 104 may be displayed superimposed on the frame 40 by using an alpha blending method.

The position at which each of the identifiers 104 is displayed in the frame 40 is a position adjacent to the corresponding lesion 42 (hereinafter also referred to as a “lesion-adjacent position”). The control unit 82C selects one of the plurality of pieces of first information 111 and refers to the second position information 109 included in selected first information, which is the selected one of the plurality of pieces of first information 111, to determine the lesion-adjacent position. Then, the control unit 82C displays the identifier 104 included in the selected first information at the lesion-adjacent position. In the present embodiment, the “lesion-adjacent position” is an example of the “second display position” according to the technology of the present disclosure.

In the second display region 38, the plurality of local images 110 included in the plurality of pieces of first information 111 acquired from the RAM 84 are displayed by the control unit 82C in accordance with the plurality of sizes 112 included in the plurality of pieces of second information 126 acquired from the RAM 84. In the present embodiment, the position at which each of the local images 110 is displayed in the second display region 38 is an example of the “first display position” according to the technology of the present disclosure.

In the second display region 38, the plurality of local images 110 included in the plurality of pieces of first information 111 acquired from the RAM 84 are displayed so as to be arranged side by side such that they can be compared. For example, in the second display region 38, the plurality of local images 110 are displayed in the vertical direction (in other words, in the up-down direction when viewed from the front).

In the second display region 38, furthermore, the plurality of local images 110 are displayed in a display style that enables the visual identification of the relationship in magnitude between the sizes 112 of the local images 110. For example, in the second display region 38, the plurality of local images 110 are arranged from top to bottom in descending order of the sizes 112.

In the second display region 38, furthermore, the plurality of sizes 112 included in the plurality of pieces of first information 111 acquired from the RAM 84 are displayed in a manner that enables the identification of the correspondence relationship with the plurality of local images 110. For example, in the second display region 38, each of the plurality of sizes 112 is displayed at a position adjacent to the corresponding local image 110.

The correspondence relationship between the local image 110 included in the first information 111 and the size 112 included in the second information 126 is identified by checking the identifier 104 included in the first information 111 against the identifier 104 included in the second information 126.

In the second display region 38, furthermore, the identifier 104 corresponding to each of the local images 110 is displayed at a position adjacent to the local image 110. This allows the doctor 12 to check the identifier 104 displayed at the lesion-adjacent position in the first display region 36 against the identifier 104 displayed in the second display region 38 to visually identify which of the lesions 42 in the frame 40 the lesion 42 appearing in each of the local images 110 corresponds to.

In the example illustrated in FIG. 11, each of the identifiers 104 is displayed to the left of the corresponding local image 110 when viewed from the front, and each of the sizes 112 is displayed to the right of the corresponding local image 110 when viewed from the front. However, this is merely an example. It is sufficient that each of the identifiers 104, each of the local images 110, and each of the sizes 112 be displayed in the second display region 38 in a layout that enables the identification of the correspondence relationship between them. In addition, it is sufficient that the plurality of local images 110 be displayed in the second display region 38 in a layout that enables the visual identification of the relationship in magnitude between the sizes 112 of the plurality of lesions 42.

While the example illustrated in FIG. 11 provides an example embodiment in which the correspondence relationship between the lesions 42 appearing in the frame 40 and the local images 110 is visually identified using the identifiers 104, this is merely an example. For example, the first rectangular frames 106 (see FIG. 7) may be displayed in the frame 40, and the second rectangular frame 108 of each of the local images 110 displayed in the second display region 38 may be displayed in the same display style (for example, color, luminance, and/or the like) as that of the first rectangular frame 106 having the correspondence relationship with the second rectangular frame 108. Alternatively, the lesion 42 in the frame 40 and the corresponding local image 110 may be displayed so as to be associated with each other through a line or the like.

While the example illustrated in FIG. 11 provides an example embodiment in which the sizes 112 are displayed in the second display region 38, this is merely an example. The sizes 112 may be displayed in the frame 40 displayed in the first display region 36. In this case, for example, the sizes 112 may be displayed superimposed on the frame 40 by using an alpha blending method.

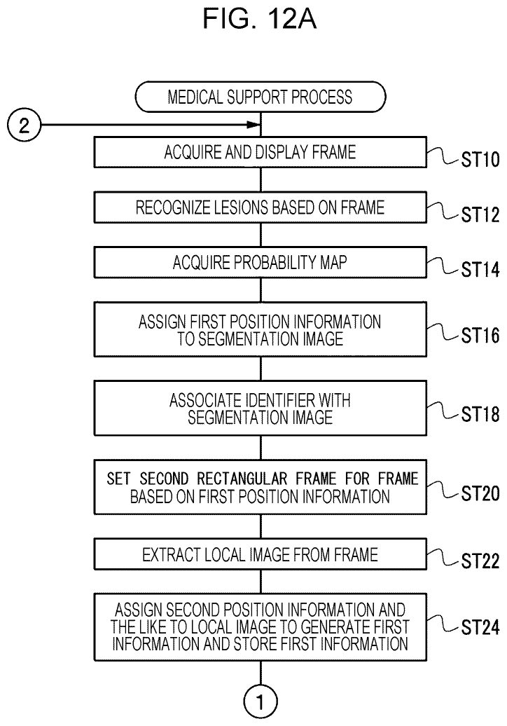

Next, the operation of a portion of the endoscope system 10 according to the technology of the present disclosure will be described with reference to FIGS. 12A and 12B. The flow of the medical support process illustrated in FIGS. 12A and 12B is an example of the “medical support method” according to the technology of the present disclosure.

In the medical support process illustrated in FIG. 12A, first, in step ST10, the recognition unit 82A and the control unit 82C acquire a frame 40 obtained by imaging the large intestine 28 with the camera 52. Then, the control unit 82C displays the frame 40 in the first display region 36 (see FIGS. 5 and 11). For convenience of description, it is assumed here that a plurality of lesions 42 appear in the frame 40. After the processing of step ST10 is performed, the medical support process proceeds to step ST12.

In step ST12, the recognition unit 82A performs the recognition process 96 on the frame 40 acquired in step ST10 to recognize the lesions 42 appearing in the frame 40 (see FIG. 5). After the processing of step ST12 is performed, the medical support process proceeds to step ST14.

In step ST14, the recognition unit 82A acquires the probability map 100 from the recognition model 92 (see FIG. 6). After the processing of step ST14 is performed, the medical support process proceeds to step ST16.

In step ST16, the recognition unit 82A assigns first position information 98 to each of the plurality of segmentation images 102 on the probability map 100 acquired in step ST14 (see FIG. 6). After the processing of step ST16 is performed, the medical support process proceeds to step ST18.

In step ST18, the recognition unit 82A assigns an identifier 104 to each of the pieces of first position information 98 each assigned to a corresponding one of the plurality of segmentation images 102 on the probability map 100 to associate the identifier 104 with the corresponding one of the plurality of segmentation images 102 (see FIG. 6). After the processing of step ST18 is performed, the medical support process proceeds to step ST20.

In step ST20, the control unit 82C sets a second rectangular frame 108 for each of a plurality of image regions indicating the plurality of lesions 42 in the frame 40, based on the pieces of first position information 98 each assigned to a corresponding one of the plurality of segmentation images 102 on the probability map 100 in step ST16 (see FIG. 7). After the processing of step ST20 is performed, the medical support process proceeds to step ST22.

In step ST22, the control unit 82C extracts a plurality of local images 110 from the frame 40 using the plurality of second rectangular frames 108 set for the frame 40 in step ST20 (see FIG. 8). After the processing of step ST22 is performed, the medical support process proceeds to step ST24.

In step ST24, the control unit 82C assigns the identifier 104 and the second position information 109 to each of the plurality of local images 110 extracted from the frame 40 to generate a plurality of pieces of first information 111, and stores the pieces of first information 111 in the RAM 84 (see FIG. 8). After the processing of step ST24 is performed, the medical support process proceeds to step ST26 illustrated in FIG. 12B.

In step ST26, the acquisition unit 82B acquires the size 112 of each of the plurality of lesions 42 appearing in the frame 40, based on the frame 40 acquired in step ST10 and the probability map 100 in which the pieces of first position information 98 are associated with the segmentation images 102 (see FIG. 9). After the processing of step ST26 is performed, the medical support process proceeds to step ST28.

In step ST28, the acquisition unit 82B associates, for each of the plurality of lesions 42 appearing in the frame 40, the size 112 acquired in step ST26 with the identifier 104 associated with the segmentation image 102 used to acquire the size 112 to generate second information 126 and stores the second information 126 in the RAM 84 (see FIG. 10). After the processing of step ST28 is performed, the medical support process proceeds to step ST30.

In step ST30, the control unit 82C displays the identifiers 104 at the lesion-adjacent positions in the frame 40, based on the pieces of first information 111 stored in the RAM 84 (see FIG. 11). After the processing of step ST30 is performed, the medical support process proceeds to step ST32.

In step ST32, the control unit 82C displays, for each of the identifiers 104, the local image 110 and the size 112 in the second display region 38, based on the first information 111 and the second information 126 stored in the RAM 84 (see FIG. 11). After the processing of step ST32 is performed, the medical support process proceeds to step ST34.

In step ST34, the control unit 82C determines whether a condition for ending the medical support process is satisfied. An example of the condition for ending the medical support process is a condition in which an instruction to end the medical support process is given to the endoscope system 10 (for example, a condition in which the instruction to end the medical support process is received by the reception device 64).

If the condition for ending the medical support process is not satisfied in step ST34, the determination is negative, and the medical support process proceeds to step ST10 illustrated in FIG. 12A. If the condition for ending the medical support process is satisfied in step ST34, the determination is affirmative, and the medical support process ends.

As described above, in the endoscope system 10, the recognition process 96 is performed on the frame 40 in which a plurality of lesions 42 appear to recognize the plurality of lesions 42, and the size 112 is acquired as a characteristic of each of the plurality of lesions 42. The frame 40 is displayed in the first display region 36, and a plurality of local images 110 in which the plurality of lesions 42 are individually extracted from the frame 40 are displayed in the second display region 38 in accordance with the respective sizes 112 of the plurality of lesions 42. This enables the doctor 12 to visually recognize the plurality of lesions 42 appearing in the frame 40 in a manner that enables the doctor 12 to grasp the respective sizes 112 of the plurality of lesions 42, without impairing the visibility of the frame 40 displayed in the first display region 36.

While an example embodiment in which the plurality of local images 110 are displayed in the second display region 38 in accordance with the sizes 112 (that is, the actual sizes) of the plurality of lesions 42 is given here, this is merely an example. For example, since differences in the sizes 112 of the plurality of lesions 42 can also be identified from differences in the sizes (that is, the sizes in the image) of the first rectangular frames 106 each set for a corresponding one of the plurality of lesions 42, the plurality of local images 110 may be displayed in the second display region 38 in accordance with the sizes of the first rectangular frames 106 each set for a corresponding one of the plurality of lesions 42. Alternatively, the plurality of local images 110 may be displayed in the second display region 38 in accordance with the sizes of the plurality of segmentation images 102 corresponding to the plurality of lesions 42.

In the endoscope system 10, furthermore, the plurality of local images 110 are displayed in the second display region 38 in a display style that enables the visual identification of the relationship in magnitude between the sizes 112 of the plurality of lesions 42. This allows the doctor 12 to visually recognize the relationship in magnitude between the sizes 112 of the plurality of lesions 42.

In the endoscope system 10, furthermore, the sizes 112 corresponding to the plurality of local images 110 are displayed in the second display region 38 in a manner that enables the identification of the correspondence relationship with the plurality of local images 110. This allows the doctor 12 to visually recognize differences in the sizes 112 of the plurality of lesions 42.

In the endoscope system 10, furthermore, the local images 110 are images extracted from the frame 40 using the second rectangular frames 108 that make differences in the sizes 112 of the lesions 42 between the plurality of local images 110 visually distinguishable when the plurality of local images 110 are compared. This allows the doctor 12 to visually recognize differences in the sizes 112 of the plurality of lesions 42.

In the endoscope system 10, furthermore, the second rectangular frames 108 are frames having a shape and a size common to the plurality of lesions 42. Accordingly, the plurality of local images 110 extracted from the frame 40 using the second rectangular frames 108 are displayed in the second display region 38 in a manner that enables comparison with each other. This allows the doctor 12 to visually recognize differences in the sizes 112 of the plurality of lesions 42.

In the endoscope system 10, furthermore, the identifiers 104 are displayed in the second display region 38 at positions adjacent to the corresponding local images 110, and the identifiers 104 are also displayed in the first display region 36 at the lesion-adjacent positions. This allows the doctor 12 to visually recognize the correspondence relationship between the display positions of the local images 110 in the second display region 38 and the display positions of the lesions 42 appearing in the frame 40 in the first display region 36.

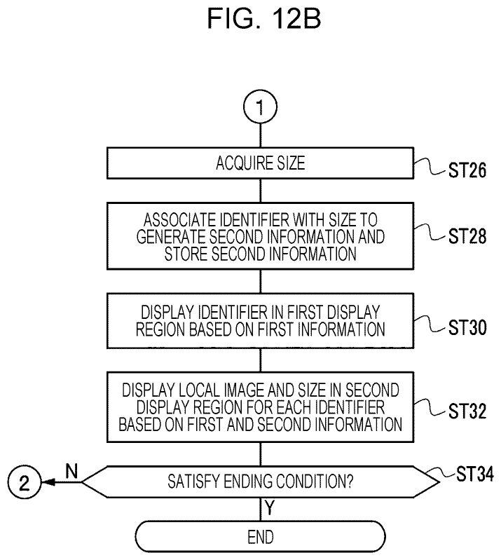

While the embodiment described above provides an example embodiment in which the acquisition unit 82B acquires the size 112 of each of the plurality of lesions 42 and an identifier 104 is associated with the size 112 to generate second information 126, the technology of the present disclosure is not limited to this. For example, as illustrated in FIG. 13, the acquisition unit 82B may generate a plurality of pieces of third information 128, based on a plurality of pieces of distance information 114 obtained for the plurality of lesions 42 and store the pieces of third information in the RAM 84. The third information 128 is different from the second information 126 in that the distance information 114 is used instead of the size 112. The distance information 114 used for the third information 128 is distance information 114 extracted from the segmentation-corresponding region 116A to determine the size 112. The distance information 114 is an example of the “depth” according to the technology of the present disclosure.

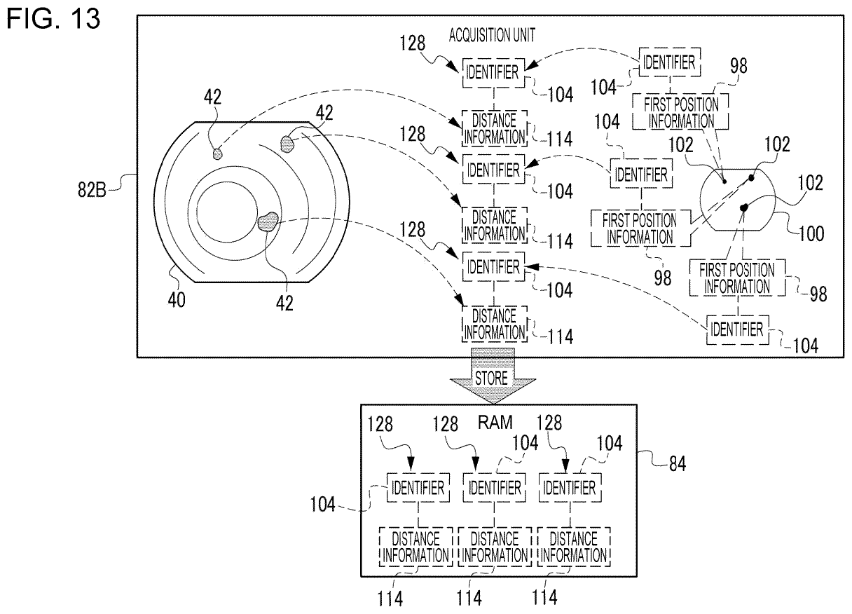

In a case where the RAM 84 stores the plurality of pieces of first information 111 and the plurality of pieces of third information 128, as an example, as illustrated in FIG. 14, the control unit 82C acquires the plurality of pieces of first information 111 and the plurality of pieces of third information 128 from the RAM 84 and displays the plurality of identifiers 104, the plurality of local images 110, and the plurality of pieces of distance information 114 in the second display region 38, based on the plurality of pieces of first information 111 and the plurality of pieces of third information 128 acquired from the RAM 84. The content displayed in the second display region 38 illustrated in FIG. 14 is different from the content displayed in the second display region 38 illustrated in FIG. 11 in that the distance information 114 is displayed instead of the size 112. That is, the second display region 38 displays the plurality of local images 110 in a display style that enables the visual identification of the relationship in depth between the plurality of lesions 42. In the example illustrated in FIG. 14, at a position adjacent to each of the local images 110, the corresponding piece of distance information 114 (that is, the depth from the camera 52 to the lesion 42 appearing in the local image 110) is displayed.

As described above, the plurality of local images 110 are displayed in the second display region 38 in accordance with the pieces of distance information 114. This allows the doctor 12 to visually recognize the plurality of lesions 42 appearing in the frame 40 in a manner that enables the doctor 12 to grasp the distance information 114 of each of the plurality of lesions 42, without impairing the visibility of the frame 40 displayed in the first display region 36. In addition, since the distance information 114 corresponding to each of the plurality of local images 110 is displayed at a position adjacent to the corresponding local image 110, the doctor 12 can visually recognize the relationship in the depth from the observation position between the plurality of lesions 42.

While the example illustrated in FIG. 14 provides an example embodiment in which the plurality of pieces of distance information 114 are displayed in the second display region 38, this is merely an example. The plurality of sizes 112 and the plurality of pieces of distance information 114 may be displayed in the second display region 38 so as to be arranged side by side.

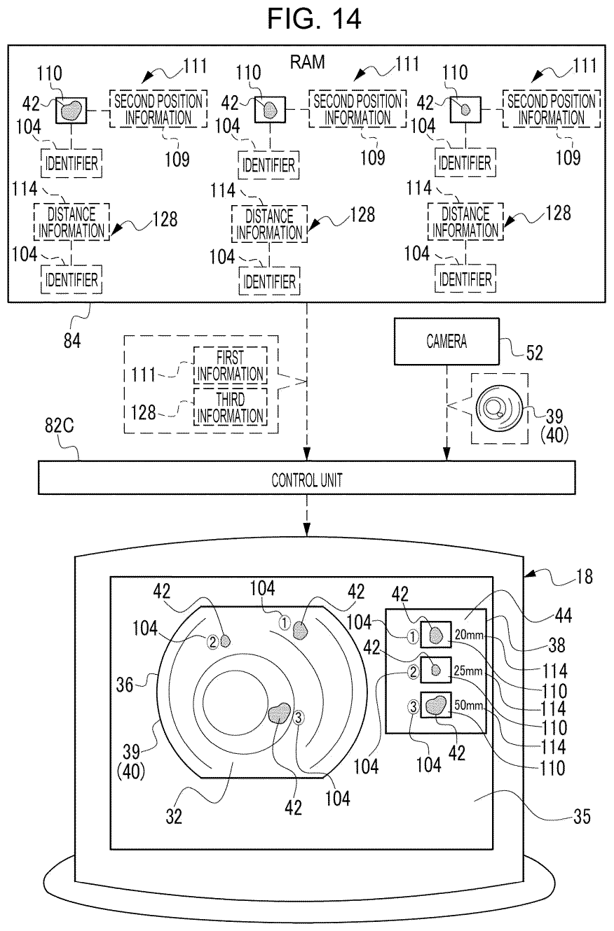

While the example illustrated in FIG. 11 provides an example embodiment in which the plurality of local images 110 are displayed in the second display region 38 in accordance with the sizes 112 and the example illustrated in FIG. 14 provides an example embodiment in which the plurality of local images 110 are displayed in the second display region 38 in accordance with the pieces of distance information 114, the content displayed in the second display region 38 illustrated in FIG. 11 and the content displayed in the second display region 38 illustrated in FIG. 14 may be selectively displayed. In this case, for example, as illustrated in FIG. 15, the content displayed in the second display region 38 illustrated in FIG. 11 and the content displayed in the second display region 38 illustrated in FIG. 14 may be switched in accordance with an instruction 129 received by the reception device 64 (for example, an instruction given by the doctor 12). This allows the doctor 12 to display the plurality of lesions 42 appearing in the frame 40 in the second display region 38 in a manner that enables the doctor 12 to grasp the respective sizes 112 of the plurality of lesions 42 or to display the plurality of lesions 42 appearing in the frame 40 in the second display region 38 in a manner that enables the doctor 12 to grasp the distance information 114 of each of the plurality of lesions 42, in accordance with the intention of the doctor 12.

As the number of lesions 42 appearing in the frame 40 increases, the number of local images 110 to be displayed in the second display region 38 also increases, and too large a number of lesions 42 causes difficulty in displaying all the local images 110 in the second display region 38. It may be possible to reduce the sizes of the local images 110 to display all the local images 110 in the second display region 38, although the visibility of the local images 110 becomes low.

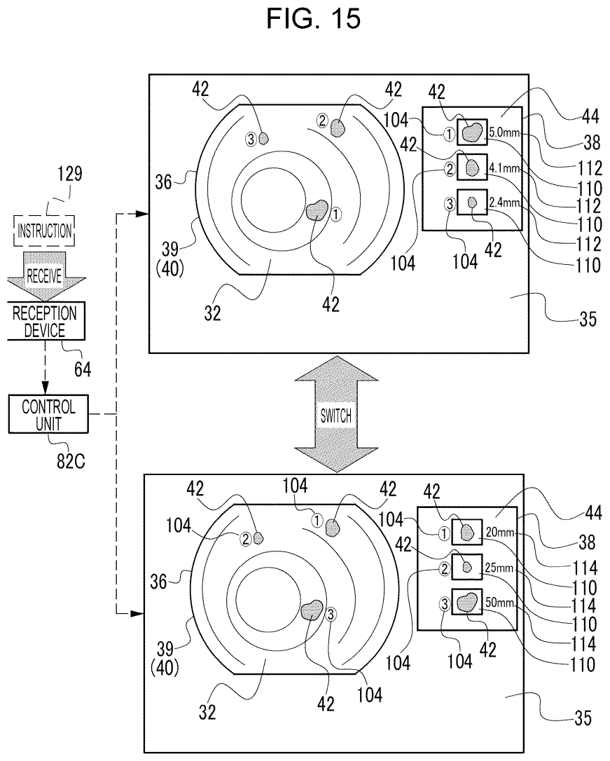

Accordingly, as an example, as illustrated in FIG. 16, the control unit 82C displays a plurality of local images 110 in the second display region 38 in such a manner as to be grouped. In the example illustrated in FIG. 16, the control unit 82C determines whether the number of local images 110 stored in the RAM 84 (that is, the number of local images 110 corresponding to the number of lesions 42 recognized by the recognition unit 82A) exceeds a predetermined number (for example, four). An example of the predetermined number is the number of local images derived in advance through a test using an actual machine, computer simulation, and/or the like as the number of local images whose visibility would be low if all the local images 110 stored in the RAM 84 are displayed in the second display region 38.

If the number of local images 110 stored in the RAM 84 does not exceed the predetermined number, the control unit 82C performs a process similar to that in the embodiment described above. If the number of local images 110 stored in the RAM 84 exceeds the predetermined number, the control unit 82C assigns the plurality of local images 110 stored in the RAM 84 to a plurality of size ranges to group the plurality of local images 110 by size range.

Examples of the plurality of size ranges include a first size range 130 and a second size range 132. For example, the first size range 130 is a range in which the size 112 is greater than or equal to 4.0 mm, and the second size range 132 is a range in which the size 112 is less than 4.0 mm. A size 112 in the range greater than or equal to 4.0 mm and a size 112 in the range less than 4.0 mm are each an example of the “common characteristic” according to the technology of the present disclosure.

While two size ranges are exemplified here for convenience of description, this is merely an example, and three or more size ranges may be used, for example, so long as the plurality of sizes 112 are classified into a plurality of size ranges.

The plurality of size ranges may be determined based on a reference value referred to by the doctor 12 for clinical decision making based on medical knowledge (for example, a reference value (for example, 5 mm, 10 mm, and/or the like) based on which the doctor 12 determines whether to excise the lesions 42), or may be determined based on a variable value that is changed in accordance with an instruction given by the doctor 12 and/or various conditions.

The control unit 82C assigns the plurality of local images 110 to the first size range 130 and the second size range 132, based on the pieces of first information 111 and the pieces of second information 126 stored in the RAM 84 to group the plurality of local images 110 by size range. Then, the control unit 82C displays the plurality of local images 110 in the second display region 38 in such a manner as to be grouped by size range. In the example illustrated in FIG. 16, a local image 110 that is representative of the first size range 130 and a local image 110 that is representative of the second size range 132 are displayed in the second display region 38. In the example illustrated in FIG. 16, furthermore, the size 112 of the lesion 42 appearing in the local image 110 representative of the first size range 130 and the size 112 of the lesion 42 appearing in the local image 110 representative of the second size range 132 are displayed in the second display region 38.

In the present embodiment, the first size range 130 and the second size range 132 are an example of the “plurality of first ranges” according to the technology of the present disclosure.

The second display region 38 displays first number-of-local-images information 134 indicating the number of local images 110 assigned to the first size range 130 and second number-of-local-images information 136 indicating the number of local images 110 assigned to the second size range 132. In the example illustrated in FIG. 16, information indicating that the number of local images 110 assigned to the first size range 130 is two is displayed as the first number-of-local-images information 134, and information indicating that the number of local images 110 assigned to the second size range 132 is three is displayed as the second number-of-local-images information 136. While specific numbers of local images are exemplified here, this is merely an example, and a schematic measure that can determine whether the number of local images is large or small may be used. In the present embodiment, the first number-of-local-images information 134 and the second number-of-local-images information 136 are examples of the “information related to the number of extracted images grouped in the first range” according to the technology of the present disclosure.

An example of the local image 110 representative of the first size range 130 is the local image 110 in which the lesion 42 having the maximum size 112 appears among all the local images 110 assigned to the first size range 130. An example of the local image 110 representative of the second size range 132 is the local image 110 in which the lesion 42 having the maximum size 112 appears among all the local images 110 assigned to the second size range 132.

While the local image 110 in which the lesion 42 having the maximum size 112 appears is exemplified here as the local image 110 representative of a size range, this is merely an example. For example, the local image 110 representative of a size range may be the local image 110 in which the lesion 42 having the minimum size 112 appears, the local image 110 in which the lesion 42 having the median size 112 appears, the local image 110 in which the lesion 42 having the mode size 112 appears, or a randomly selected local image 110.

In the example illustrated in FIG. 16, furthermore, the identifier 104 corresponding to the local image 110 representative of the first size range 130 is displayed in the second display region 38, and the identifier 104 corresponding to the local image 110 representative of the second size range 132 is displayed in the second display region 38, in a manner similar to that in the embodiment described above.

In the example illustrated in FIG. 16, as described above, the plurality of sizes 112 of the plurality of lesions 42 appearing in the plurality of local images 110 are classified into the first size range 130 and the second size range 132, and the plurality of local images 110 assigned to the first size range 130 and the plurality of local images 110 assigned to the second size range 132 are displayed in groups in the second display region 38. This achieves higher visibility of the second display region 38 than when all the local images 110 are separately displayed in the second display region 38.

In the example illustrated in FIG. 16, furthermore, a local image 110 that is a representative of all the local images 110 assigned to the first size range 130 is displayed in the second display region 38, and the first number-of-local-images information 134 is displayed in the second display region 38 as information related to the number of local images 110 grouped in the first size range 130. Further, a local image 110 that is a representative of all the local images 110 assigned to the second size range 132 is displayed in the second display region 38, and the second number-of-local-images information 136 is displayed in the second display region 38 as information related to the number of local images 110 grouped in the second size range 132. This allows the doctor 12 to approximately grasp the difference between the first size range 130 and the second size range 132.

In the example illustrated in FIG. 16, furthermore, when the number of local images 110 stored in the RAM 84 exceeds the predetermined number, the plurality of local images 110 are displayed in the second display region 38 in such a manner as to be grouped by size range. Accordingly, with this configuration, a number of local images 110 that would not interfere with visibility can be displayed in the second display region 38, whereas a number of local images 110 that would interfere with visibility can be grouped and displayed in the second display region 38. As a result, visual discomfort experienced by the doctor 12 observing the second display region 38 can be reduced.

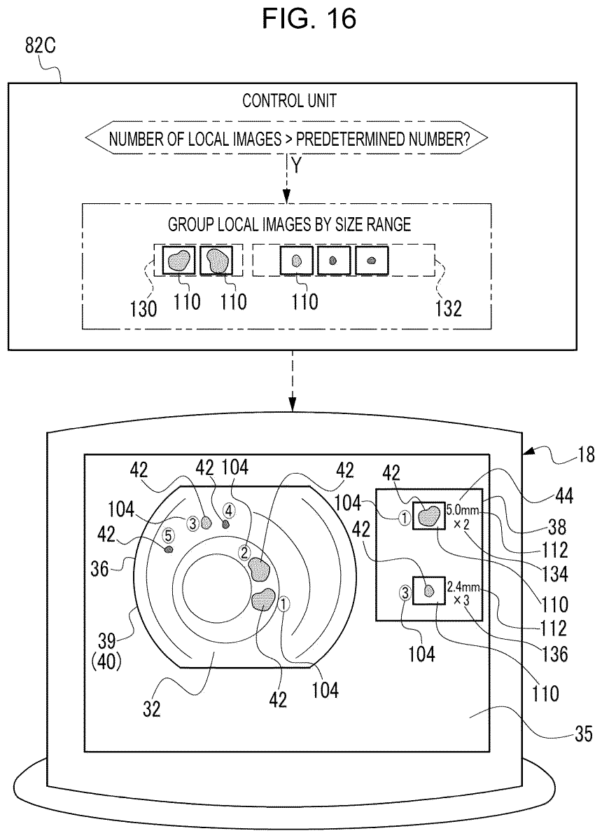

While the example illustrated in FIG. 16 provides an example embodiment in which the plurality of local images 110 are grouped by size range, the technology of the present disclosure is not limited to this. For example, as illustrated in FIG. 17, the plurality of local images 110 may be grouped by distance range.

The example illustrated in FIG. 17 is different from the example illustrated in FIG. 16 in that a first distance range 138 is used instead of the first size range 130 and a second distance range 140 is used instead of the second size range 132. In the example illustrated in FIG. 14, the pieces of distance information 114 are displayed in the second display region 38, whereas in the example illustrated in FIG. 17, instead of the pieces of distance information 114, indicators 141 indicating the depths of the lesions 42 are displayed in the second display region 38. Each of the indicators 141 is represented as “deep” or “shallow”. While the example illustrated in FIG. 17 provides an example embodiment in which the indicators 141 are displayed in the second display region 38, this is merely an example. The pieces of distance information 114 of the lesions 42 appearing in the local images 110 representative of the respective distance ranges may be displayed in the second display region 38 in a manner similar to that in which the sizes 112 of the lesions 42 appearing in the local images 110 representative of the respective size ranges are displayed in the second display region 38 in the example illustrated in FIG. 16. If the number of local images 110 stored in the RAM 84 exceeds the predetermined number, the control unit 82C assigns the plurality of local images 110 stored in the RAM 84 to a plurality of distance ranges to group the plurality of local images 110 by distance range. Examples of the plurality of distance ranges include the first distance range 138 and the second distance range 140. For example, the first distance range 138 is a range in which the distance indicated by the distance information 114 is greater than or equal to 4.0 mm, and the second distance range 140 is a range in which the distance indicated by the distance information 114 is less than 4.0 mm. A distance in the range greater than or equal to 4.0 mm and a distance in the range less than 4.0 mm are each an example of the “common characteristic” according to the technology of the present disclosure.

While two distance ranges are exemplified here for convenience of description, this is merely an example, and three or more distance ranges may be used, for example, so long as the plurality of pieces of distance information 114 are classified into a plurality of distance ranges. The plurality of distance ranges may be fixed values or variable values that are changed in accordance with an instruction given by the doctor 12 and/or various conditions.

The control unit 82C assigns the plurality of local images 110 to the first distance range 138 and the second distance range 140, based on the pieces of first information 111 and the pieces of third information 128 (see FIG. 14) stored in the RAM 84 to group the plurality of local images 110 by distance range. Then, the control unit 82C displays the plurality of local images 110 in the second display region 38 in such a manner as to be grouped by distance range. In the example illustrated in FIG. 17, a local image 110 that is representative of the first distance range 138 and a local image 110 that is representative of the second distance range 140 are displayed in the second display region 38. In the present embodiment, the first distance range 138 and the second distance range 140 are an example of the “plurality of second ranges” according to the technology of the present disclosure.

The second display region 38 displays an indicator 141 indicating the depth of the lesion 42 appearing in the local image 110 assigned to the first distance range 138, and an indicator 141 indicating the depth of the lesion 42 appearing in the local image 110 assigned to the second distance range 140. The second display region 38 further displays third number-of-local-images information 142 indicating the number of local images 110 assigned to the first distance range 138 and fourth number-of-local-images information 144 indicating the number of local images 110 assigned to the second distance range 140. In the example illustrated in FIG. 17, information indicating that the number of local images 110 assigned to the first distance range 138 is two is displayed as the third number-of-local-images information 142, and information indicating that the number of local images 110 assigned to the second distance range 140 is three is displayed as the fourth number-of-local-images information 144. In the present embodiment, the third number-of-local-images information 142 and the fourth number-of-local-images information 144 are examples of the “information related to the number of extracted images grouped in the second range” according to the technology of the present disclosure.

An example of the local image 110 representative of the first distance range 138 is the local image 110 in which the lesion 42 having the maximum distance indicated by the distance information 114 appears among all the local images 110 assigned to the first distance range 138. An example of the local image 110 representative of the second distance range 140 is the local image 110 in which the lesion 42 having the maximum distance indicated by the distance information 114 appears among all the local images 110 assigned to the second distance range 140.

While the local image 110 in which the lesion 42 having the maximum distance appears is exemplified here as the local image 110 representative of a distance range, this is merely an example. For example, the local image 110 representative of a distance range may be the local image 110 in which the lesion 42 having the minimum distance appears, the local image 110 in which the lesion 42 having the median distance appears, the local image 110 in which the lesion 42 having the mode distance appears, or a randomly selected local image 110.

In the example illustrated in FIG. 17, furthermore, the identifier 104 corresponding to the local image 110 representative of the first distance range 138 is displayed in the second display region 38, and the identifier 104 corresponding to the local image 110 representative of the second distance range 140 is displayed in the second display region 38, in a manner similar to that in the embodiment described above.

In the example illustrated in FIG. 17, as described above, the plurality of pieces of distance information 114 of the plurality of lesions 42 appearing in the plurality of local images 110 are classified into the first distance range 138 and the second distance range 140, and the plurality of local images 110 assigned to the first distance range 138 and the plurality of local images 110 assigned to the second distance range 140 are displayed in groups in the second display region 38. This achieves higher visibility of the second display region 38 than when all the local images 110 are separately displayed in the second display region 38.

In the example illustrated in FIG. 17, furthermore, a local image 110 that is a representative of all the local images 110 assigned to the first distance range 138 is displayed in the second display region 38, and the third number-of-local-images information 142 is displayed in the second display region 38 as information related to the number of local images 110 grouped in the first distance range 138. Further, a local image 110 that is a representative of all the local images 110 assigned to the second distance range 140 is displayed in the second display region 38, and the fourth number-of-local-images information 144 is displayed in the second display region 38 as information related to the number of local images 110 grouped in the second distance range 140. This allows the doctor 12 to approximately grasp the difference between the first distance range 138 and the second distance range 140.

In the example illustrated in FIG. 17, furthermore, when the number of local images 110 stored in the RAM 84 exceeds the predetermined number, the plurality of local images 110 are displayed in the second display region 38 in such a manner as to be grouped by distance range. Accordingly, with this configuration, a number of local images 110 that would not interfere with visibility can be displayed in the second display region 38, whereas a number of local images 110 that would interfere with visibility can be grouped and displayed in the second display region 38. As a result, visual discomfort experienced by the doctor 12 observing the second display region 38 can be reduced.

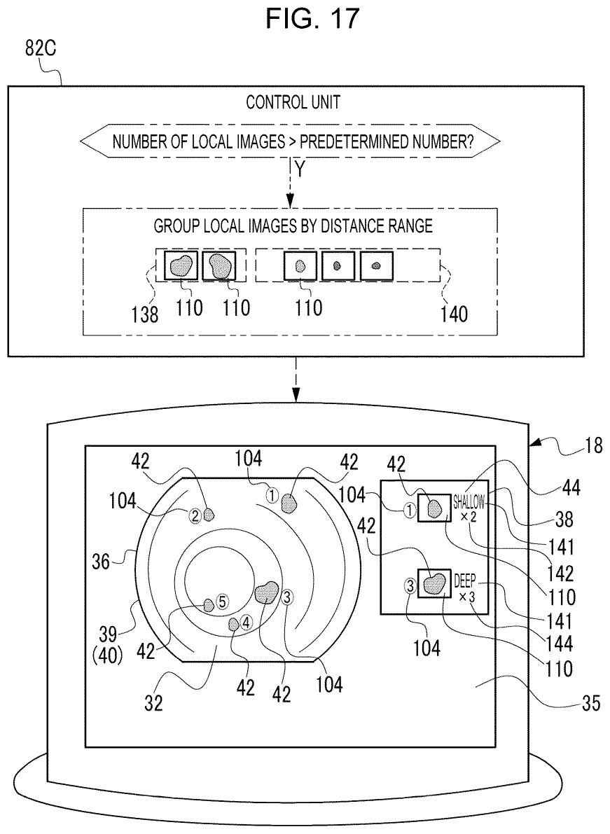

As an example, as illustrated in FIG. 18, the control unit 82C may generate a map 146 that enables the identification of the positions at which the lesions 42 included in the local images 110 are displayed in the frame 40, and display the generated map 146 in a third display region 148. The map 146 is generated by associating an identifier 104 with each of the plurality of segmentation images 102 included in the probability map 100. The respective identifiers 104 associated with the plurality of segmentation images 102 are the same identifiers as the identifiers 104 illustrated in FIG. 7.

The third display region 148 on the screen 35 is a display region different from the first display region 36 and the second display region 38 and is arranged at a position that enables comparison with the first display region 36 and the second display region 38 on the screen 35. The third display region 148 displays the probability map 100, and the plurality of identifiers 104 are displayed on the probability map 100. For example, the plurality of identifiers 104 are displayed superimposed on the probability map 100. The positions at which the identifiers 104 is displayed on the probability map 100 is a position adjacent to the segmentation images 102 and is determined based on the pieces of first position information 98 (see FIG. 7). In the present embodiment, the map 146 is an example of the “position identification information” and the “map” according to the technology of the present disclosure. In the present embodiment, the third display region 148 is an example of the “third display region” according to the technology of the present disclosure.

In the example illustrated in FIG. 18, as described above, the map 146 is displayed in the third display region 148. Since the plurality of segmentation images 102 are distributed on the map 146 at positions where the plurality of lesions 42 are present, the doctor 12 can visually identify the positions at which the lesions 42 included in the local images 110 are displayed in the frame 40 by referring to the map 146.

The map 146 is generated based on the probability map 100 obtained by performing the recognition process 96. Thus, a map that enables the identification of the positions at which the lesions 42 included in the local images 110 are displayed in the frame 40 can be easily obtained.

The map 146 is displayed in the third display region 148, which is different from the first display region 36 and the second display region 38. Thus, the visibility of the first display region 36 and the second display region 38 can be kept higher than when the map 146 is displayed in the first display region 36 or the map 146 is displayed in the second display region 38.

While the example illustrated in FIG. 18 provides an example embodiment in which the map 146 is displayed in the third display region 148, the third display region 148 may display the probability map 100 itself, a map obtained by processing the probability map 100, or the like.

While the map 146 includes the plurality of segmentation images 102, instead of the use of the plurality of segmentation images 102 on the map 146, the identifiers 104 corresponding to the segmentation images 102 may be displayed at the respective positions of the plurality of segmentation images 102 on the map 146. In this case, instead of the probability map 100, a thumbnail image of the frame 40 or an image with an outer edge having geometric similarity to the outer edge of the frame 40 may be used.

Furthermore, the shapes, the sizes, and/or the positions of the first display region 36, the second display region 38, and the third display region 148 on the screen 35 may be changed in accordance with a given instruction and/or various conditions.

One or two of the information displayed in the first display region 36, the information displayed in the second display region 38, and the information displayed in the third display region 148 may be displayed on one or more display devices different from the display device 18.

While the embodiment described above provides an example embodiment in which, for each size 112, a local image 110 is displayed in the second display region 38, the technology of the present disclosure is not limited to this. For example, the local images 110 may be assigned to the sizes 112 and the pieces of distance information 114 and displayed in the second display region 38. Alternatively, the local images 110 may be assigned to the types of the lesions 42 and displayed in the second display region 38, or the local images 110 may be assigned to the categories of the lesions 42 and displayed in the second display region 38. Alternatively, the local images 110 may be assigned to the types of the lesions 42 and/or the categories of the lesions 42 and to the sizes 112 and displayed in the second display region 38. Alternatively, the local images 110 may be assigned to the types of the lesions 42 and/or the categories of the lesions 42 and to the pieces of distance information 114 and displayed in the second display region 38.

Alternatively, the local images 110 may be assigned to characteristics (for example, the sizes 112, the pieces of distance information 114, the types of the lesions 42, and/or the categories of the lesions 42 described above) selected in accordance with an instruction received by the reception device 64 (for example, an instruction given by the doctor 12) and displayed in the second display region 38.

While the size 112, the distance information 114, the type of the lesion 42, and the category of the lesion 42 are exemplified as the characteristics of each of the lesions 42, this is merely an example. The characteristics of each of the lesions 42 may include the severity of the lesion 42, the state of the mucous membrane of the lesion 42, and/or the like, or may be characteristics obtained by combining the plurality of characteristics described above (for example, characteristics obtained by combining two or more characteristics among the size 112, the distance information 114, the type of the lesion 42, the category of the lesion 42, the severity of the lesion 42, the state of the mucous membrane of the lesion 42, and the like).

While the embodiment described above provides an example embodiment in which the identifiers 104 and the sizes 112 are displayed on the screen 35, the technology of the present disclosure is not limited to this. The technology of the present disclosure is also applicable when the identifiers 104 and/or the sizes 112 are not displayed on the screen 35. While the example illustrated in FIG. 14 provides an example embodiment in which the identifiers 104 and the pieces of distance information 114 are displayed on the screen 35, the technology of the present disclosure is also applicable when the identifiers 104 and/or the pieces of distance information 114 are not displayed on the screen 35.

While the embodiment described above provides an example embodiment in which images cropped from the frame 40 using the second rectangular frames 108 are used as local images 110, the technology of the present disclosure is not limited to this. For example, the local images 110 may be images obtained by performing image processing (for example, commonly known image processing) on images cropped from the frame 40 using the second rectangular frames 108. Alternatively, an image cropped from the frame 40 using the largest first rectangular frame 106 among all the first rectangular frames 106 (see FIG. 7) set for the probability map 100 may be used as a local image 110.

While the embodiment described above exemplifies the AI-based object recognition process using a segmentation method as the recognition process 96, the technology of the present disclosure is not limited to this, and the recognition process 96 may be an AI-based object recognition process using a bounding box method. In this case, for example, bounding boxes are used instead of the segmentation images 102 (see FIG. 5), and the bounding boxes are used as frames corresponding to the first rectangular frames 106 (see FIG. 7).

While the embodiment described above provides an example embodiment in which the endoscopic moving image 39 is displayed in the first display region 36, a result of the recognition process 96 performed on the endoscopic moving image 39 may be displayed superimposed on the endoscopic moving image 39 in the first display region 36. At least a portion of a segmentation image 102 obtained as a result of the recognition process 96 performed on the endoscopic moving image 39 may be displayed superimposed on the endoscopic moving image 39. An example in which at least a portion of the segmentation image 102 is displayed superimposed on the endoscopic moving image 39 is an example embodiment in which the outer contour of the segmentation image 102 is displayed superimposed on the endoscopic moving image 39 by using an alpha blending method.

In addition, for example, when the recognition process 96 is performed using an AI-based bounding box method, a bounding box may be displayed superimposed on the endoscopic moving image 39 in the first display region 36. Furthermore, for example, when a plurality of lesions 42 appear in the endoscopic moving image 39, at least a portion of the segmentation image 102 and/or a bounding box may be displayed superimposed in the first display region 36 as information that can visually identify which of the lesions 42 the measured size 112 corresponds to.

While the embodiment described above provides an example embodiment in which the control unit 82C generates the distance image 116 (see FIG. 9) from the frame 40 by using the distance derivation model 94 (see FIG. 9), the technology of the present disclosure is not limited to this. For example, a depth sensor (for example, a sensor that measures a distance using a laser ranging method, a phase difference method, and/or the like) provided in the tip portion 50 (see FIG. 2) may measure the depth of the large intestine 28 in the depth direction, and the processor 82 may generate the distance image 116, based on the measured depth. While the embodiment described above provides an example embodiment in which the length, in real space, of the longest range across the lesion 42 along the line segment 120 is measured as the size 112, the technology of the present disclosure is not limited to this. For example, the length, in real space, of the range corresponding to the longest line segment parallel to the short sides of the rectangular frame 122 for the image region indicating the lesion 42 may be measured as the size 112 and displayed on the screen 35. In this case, the doctor 12 can grasp the length, in real space, of the longest range across the lesion 42 along the longest line segment parallel to the short sides of the rectangular frame 122 for the image region indicating the lesion 42.

The actual size of the lesion 42 may be measured in relation to the radius and/or diameter of a circumcircle of the image region indicating the lesion 42 and displayed on the screen 35. In this case, the doctor 12 can grasp the actual size of the lesion 42 in relation to the radius and/or diameter of the circumcircle of the image region indicating the lesion 42.

While the embodiment described above provides an example embodiment in which the sizes 112 are displayed in the second display region 38, this is merely an example. The sizes 112 may be displayed outside the second display region 38 in a pop-up manner from within the second display region 38, or the sizes 112 may be displayed in a region other than the second display region 38 on the screen 35. The types of lesions, the categories of lesions, and/or the like may also be displayed in the first display region 36 and/or the second display region 38, or may be displayed on a screen other than the screen 35.

While the embodiment described above provides an example embodiment in which the sizes 112 are measured frame by frame, this is merely an example, and the sizes 112 may be measured in units of multiple frames. Alternatively, representative sizes (for example, mean values, median values, maximum values, minimum values, deviations, standard deviations, mode values, and/or the like) obtained by measuring the sizes 112 in units of multiple frames may be used for the pieces of second information 126 (see FIGS. 10 and 11).

In the embodiment described above, the AI-based object recognition process is exemplified as the recognition process 96. However, the technology of the present disclosure is not limited to this. The recognition unit 82A may recognize the lesions 42 appearing in the frame 40 by the execution of a non-AI-based object recognition process (for example, template matching or the like).

While the embodiment described above describes an example embodiment in which the arithmetic expression 124 is used to calculate each of the sizes 112, the technology of the present disclosure is not limited to this. Each of the sizes 112 may be measured by performing an AI-based process on the frame 40. In this case, for example, a trained model is used that, in response to an input of the frame 40 including the lesions 42, outputs the sizes 112 of the lesions 42. To generate the trained model, deep learning is performed on a neural network by using training data in which lesions appearing in images used as example data are assigned annotations indicating the sizes of the lesions as ground-truth data.

While the embodiment described above describes an example embodiment in which the distance information 114 is derived using the distance derivation model 94, the technology of the present disclosure is not limited to this. Other methods for deriving the distance information 114 using an AI-based method include, for example, a method for combining segmentation and depth estimation (for example, regression learning to provide the distance information 114 to the entire image (for example, all the pixels constituting the image) or unsupervised learning to learn the distance of the entire image in an unsupervised way).

While the embodiment described above exemplifies the endoscopic moving image 39, the technology of the present disclosure is not limited to this. The technology of the present disclosure is also applicable to a medical moving image (for example, a moving image obtained by a modality (for example, a radiographic diagnostic apparatus, an ultrasound diagnostic apparatus, or the like) other than the endoscope system 10, such as a radiographic moving image or an ultrasound moving image) other than the endoscopic moving image 39.

While the embodiment described above provides an example embodiment in which the sizes 112 of the lesions 42 appearing in a moving image are measured, this is merely an example. The technology of the present disclosure is also applicable to the measurement of the sizes 112 of the lesions 42 appearing in a stop-motion image or a still image.

While the embodiment described above provides an example embodiment in which the distance information 114 extracted from the segmentation-corresponding region 116A in the distance image 116 is input to the arithmetic expression 124, the technology of the present disclosure is not limited to this. For example, it is sufficient that the distance information 114 corresponding to the position identified from the first position information 98 be extracted from among all the pieces of distance information 114 output from the distance derivation model 94, without the generation of the distance image 116, and the extracted distance information 114 be input to the arithmetic expression 124.

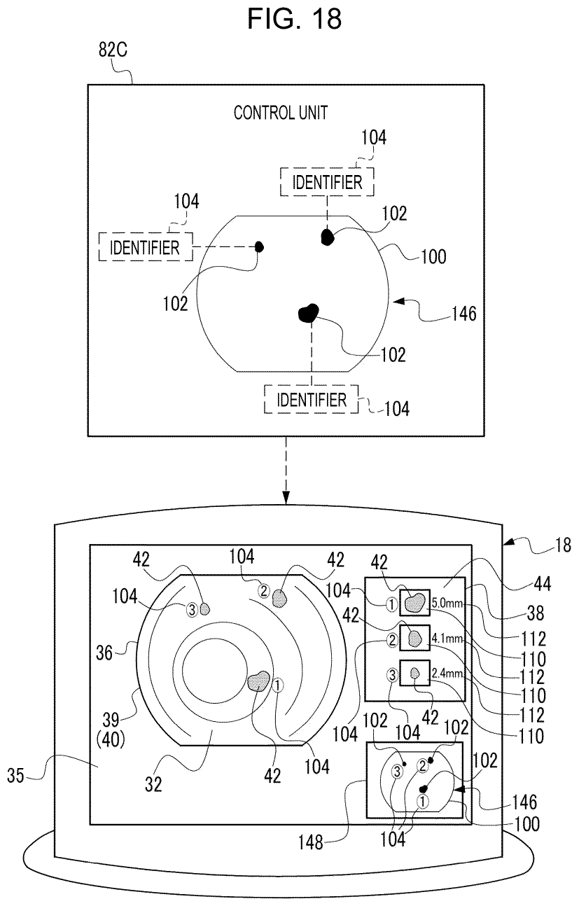

In the examples described above, the sizes 112 and the like are output to the display device 18, by way of example. However, the technology of the present disclosure is not limited to this, and various kinds of information such as the frame 40, the medical information 44, and/or the map 146 (hereinafter referred to as “various kinds of information”) may be output to a device other than the display device 18. As an example, as illustrated in FIG. 19, information that can be output as audio among the various kinds of information may be output to an audio playback device 150 as a destination. The information that can be output as audio among the various kinds of information may be output as audio by the audio playback device 150. The various kinds of information may be output to a printer 152, an electronic medical record management device 154, and/or the like as a destination. The various kinds of information may be printed as text or the like on a medium (for example, a sheet) or the like by the printer 152, or may be stored in an electronic medical record 156 managed by the electronic medical record management device 154.

While the examples described above describe an example embodiment in which the various kinds of information are displayed on the screen 35 or the various kinds of information are not displayed on the screen 35, the display of the various kinds of information on the screen 35 means the display of the various kinds of information in a manner perceptible to the user or the like (for example, the doctor 12). The concept that the various kinds of information are not displayed on the screen 35 also includes a concept of reducing the display level of the various kinds of information (for example, the level perceived through the display). For example, the concept that the various kinds of information are not displayed on the screen 35 also includes a concept of displaying the various kinds of information in a display style that does not allow the user or the like to visually perceive the various kinds of information. Examples of the display style in this case include display styles in which the various kinds of information are displayed in a reduced font size, the various kinds of information are depicted as thin lines, the various kinds of information are depicted as broken lines, the various kinds of information blink, the various kinds of information are displayed for an imperceptible period of display time, and the various kinds of information are made transparent to an imperceptible level. The same applies to the various outputs described above, such as audio output, printing, and storage.

While the embodiment described above provides an example embodiment in which the medical support process is performed by the processor 82 included in the endoscope system 10, the technology of the present disclosure is not limited to this, and a device external to the endoscope system 10 may perform at least some of the processing operations included in the medical support process.

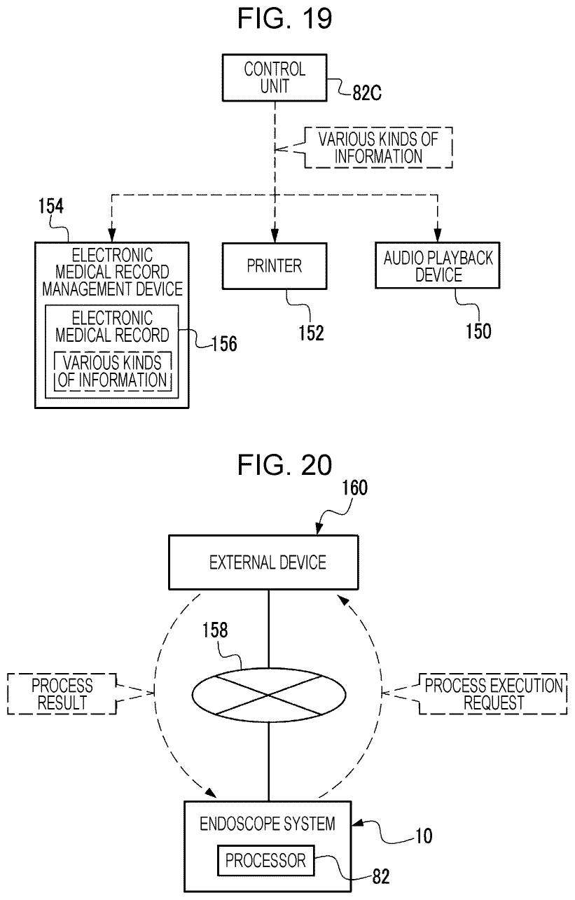

In this case, for example, as illustrated in FIG. 20, an external device 160 connected to the endoscope system 10 in a communicable manner via a network 158 (for example, a WAN, a LAN, and/or the like) is used.

Examples of the external device 160 include at least one server that transmits and receives data to and from the endoscope system 10 directly or indirectly via the network 158.

The external device 160 receives a process execution instruction given from the processor 82 of the endoscope system 10 via the network 158. Then, the external device 160 executes a process corresponding to the received process execution instruction, and transmits a process result to the endoscope system 10 via the network 158. In the endoscope system 10, the processor 82 receives the process result transmitted from the external device 160 via the network 158 and executes a process using the received process result.

Examples of the process execution instruction include an instruction to cause the external device 160 to execute at least a portion of the medical support process. A first example of at least a portion of the medical support process (that is, a process to be executed by the external device 160) is the recognition process 96. In this case, the external device 160 executes the recognition process 96 in accordance with the process execution instruction given from the processor 82 of the endoscope system 10 via the network 158, and transmits a recognition process result (for example, the first position information 98, the probability map 100, and/or the like) to the endoscope system 10 via the network 158. In the endoscope system 10, the processor 82 receives the recognition process result and executes a process similar to that in the embodiment described above by using the received recognition process result.

A second example of at least a portion of the medical support process (that is, a process to be executed by the external device 160) is a process performed by the acquisition unit 82B. The process performed by the acquisition unit 82B refers to, for example, a process for measuring the sizes 112 of the lesions 42. In this case, the external device 160 executes the process performed by the acquisition unit 82B in accordance with the process execution instruction given from the processor 82 of the endoscope system 10 via the network 158, and transmits a measurement process result (for example, the sizes 112 or the like) to the endoscope system 10 via the network 158. In the endoscope system 10, the processor 82 receives the measurement process result and executes a process similar to that in the embodiment described above by using the received measurement process result.

A third example of at least a portion of the medical support process (that is, a process to be executed by the external device 160) is at least one of the processing operations of steps ST12 to ST28 included in the medical support process illustrated in FIGS. 12A and 12B.

A fourth example of at least a portion of the medical support process (that is, a process to be executed by the external device 160) is a process for generating the third information 128 and storing the third information 128 in a storage area.

A fifth example of at least a portion of the medical support process (that is, a process to be executed by the external device 160) is a process for grouping the plurality of local images 110 by size range.

A sixth example of at least a portion of the medical support process (that is, a process to be executed by the external device 160) is a process for grouping the plurality of local images 110 by distance range.

A seventh example of at least a portion of the medical support process (that is, a process to be executed by the external device 160) is a process for generating the content to be displayed in the first display region 36, the content to be displayed in the second display region 38, and/or the content to be displayed in the third display region 148.

For example, the external device 160 is implemented by cloud computing. The cloud computing is merely an example, and the external device 160 may be implemented by network computing such as fog computing, edge computing, or grid computing. Instead of the server, at least one personal computer or the like may be used as the external device 160. Alternatively, the external device 160 may be an arithmetic device with a communication function mounted with a plurality of types of AI functions.

While the embodiment described above provides an example embodiment in which the medical support program 90 is stored in the NVM 86, the technology of the present disclosure is not limited to this. For example, the medical support program 90 may be stored in a portable non-transitory computer-readable storage medium such as an SSD or a USB memory. The medical support program 90 stored in the non-transitory storage medium is installed in the computer 78 of the endoscope system 10. The processor 82 executes the medical support process in accordance with the medical support program 90.

Alternatively, the medical support program 90 may be stored in a storage device of another computer, a server, or the like connected to the endoscope system 10 via a network, and the medical support program 90 may be downloaded in response to a request from the endoscope system 10 and installed in the computer 78.

Not all, but a portion, of the medical support program 90 may be stored in a storage device of another computer, a server device, or the like connected to the endoscope system 10, or not all, but a portion, of the medical support program 90 may be stored in the NVM 86.

Examples of a hardware resource that executes the medical support process may include the following various processors. The processors include, for example, a CPU that is a general-purpose processor configured to execute software, that is, a program, to function as a hardware resource that executes the medical support process. The processors further include, for example, a dedicated electric circuit that is a processor having a circuit configuration designed specifically for executing specific processing, such as an FPGA, a PLD, or an ASIC.

Each of the processors incorporates or is connected to a memory, and uses the memory to execute the medical support process.

The hardware resource that executes the medical support process may be configured as one of the various processors or as a combination of two or more processors of the same type or different types (for example, a combination of a plurality of FPGAs or a combination of a CPU and an FPGA). The hardware resource that executes the medical support process may be a single processor.

Examples of configuring the hardware resource as a single processor include, first, a form in which a single processor is configured as a combination of one or more CPUs and software and the processor functions as a hardware resource that executes the medical support process. The examples include, second, a form in which, as typified by an SoC or the like, a processor is used in which the functions of the entire system including a plurality of hardware resources that execute the medical support process are implemented as one IC chip. As described above, the medical support process is implemented by using one or more of the various processors described above as hardware resources.

More specifically, the hardware structure of these various processors may be an electric circuit in which circuit elements such as semiconductor elements are combined. The medical support process described above is merely an example. Thus, it goes without saying that unnecessary steps may be deleted, new steps may be added, or the processing order may be changed without departing from the gist.

The description and drawings presented above provide detailed descriptions of portions according to the technology of the present disclosure and are merely examples of the technology of the present disclosure. For example, the descriptions related to the configurations, functions, operations, and effects described above are descriptions related to an example of the configurations, functions, operations, and effects of portions according to the technology of the present disclosure. Thus, it goes without saying that unnecessary portions may be deleted or new elements may be added or substituted in the description and drawings presented above without departing from the gist of the technology of the present disclosure. To avoid complexity and facilitate understanding of portions according to the technology of the present disclosure, descriptions related to common general technical knowledge and the like, for which no specific explanation is required to implement the technology of the present disclosure, are omitted in the description and drawings presented above.

As used herein, “A and/or B” is synonymous with “at least one of A or B”. That is, “A and/or B” means only A, only B, or a combination of A and B. In this specification, furthermore, a concept similar to that of “A and/or B” is applied also to the expression of three or more matters in combination with “and/or”.

All publications, patent applications, and technical standards described herein are incorporated herein by reference to the same extent as if each individual publication, patent application, and technical standard were specifically and individually indicated to be incorporated by reference.

What is claimed is:

1. A medical support device comprising a processor,
wherein the processor is configured to:
acquire a characteristic of each of a plurality of observation target regions, the plurality of observation target regions being recognized by performing a recognition process on a medical image in which the plurality of observation target regions appear;
display the medical image in a first display region; and
display, in accordance with the characteristic, a plurality of extracted images in a second display region outside the first display region, the plurality of extracted images being images in which the plurality of observation target regions have been individually extracted from the medical image.

2. The medical support device according to claim 1, wherein the characteristic includes a size.

3. The medical support device according to claim 2, wherein the plurality of extracted images are displayed in the second display region in a display style that enables visual identification of a magnitude relationship of the size among the plurality of observation target regions.

4. The medical support device according to claim 2, wherein the size is divided into a plurality of first ranges, and the plurality of extracted images are displayed in the second display region in a state in which the plurality of extracted images are grouped by the first ranges.

5. The medical support device according to claim 4, wherein in a case in which the plurality of extracted images are grouped by the first ranges, an extracted image representative of the first range is displayed, and information regarding a number of the extracted images grouped in the first range is displayed.

6. The medical support device according to claim 4, wherein in a case in which a number of the plurality of extracted images exceeds a predetermined number, the plurality of extracted images are displayed in the second display region in a state in which the plurality of extracted images are grouped by the first ranges.

7. The medical support device according to claim 1, wherein the characteristic includes a depth.

8. The medical support device according to claim 7, wherein the plurality of extracted images are displayed in the second display region in a display style that enables visual identification of a shallowness/depth relationship of the depth among the plurality of observation target regions.

9. The medical support device according to claim 7, wherein the depth is divided into a plurality of second ranges, and the plurality of extracted images are displayed in the second display region in a state in which the plurality of extracted images are grouped by the second ranges.

10. The medical support device according to claim 9, wherein in a case in which the plurality of extracted images are grouped by the second ranges, the extracted image representative of the second range is displayed in the second display region, and information regarding a number of the extracted images grouped in the second range is displayed in the second display region.

11. The medical support device according to claim 9, wherein in a case in which a number of the plurality of extracted images exceeds a predetermined number, the plurality of extracted images are displayed in the second display region in a state in which the plurality of extracted images are grouped by the second ranges.

12. The medical support device according to claim 1, wherein the processor is configured to display positional relationship identification information on a screen, and the positional relationship identification information is information enabling identification that a first display position at which at least one of the plurality of extracted images is displayed and a second display position at which an observation target region appearing in the extracted image displayed at the first display position is displayed in the first display region are in correspondence.

13. The medical support device according to claim 1, wherein
the plurality of extracted images are displayed in the second display region in a state in which the plurality of extracted images are grouped by the common characteristic.

14. The medical support device according to claim 13, wherein
in a case in which the plurality of extracted images are grouped by the common characteristic, for each common characteristic, the extracted image representative of the characteristic is displayed in the second display region, and information regarding a number of the extracted images grouped in the common characteristic is displayed in the second display region.

15. The medical support device according to claim 13, wherein
in a case in which a number of the plurality of extracted images exceeds a predetermined number, the plurality of extracted images are displayed in the second display region in a state in which the plurality of extracted images are grouped by the common characteristic.

16. The medical support device according to claim 1, wherein
the processor is configured to display, in a third display region, position identification information enabling identification of display positions, in the medical image, of the observation target regions included in the extracted images.

17. The medical support device according to claim 16, wherein
the position identification information is a map enabling identification of the display positions in the medical image.

18. The medical support device according to claim 17, wherein
the recognition process is an object recognition process using machine learning, and
the map is generated based on a probability map obtained by performing the object recognition process.

19. The medical support device according to claim 16, wherein
the third display region is located at a different position from the first display region and the second display region.

20. The medical support device according to claim 1, wherein
respective actual sizes of the plurality of observation target regions are measured, and
the actual sizes corresponding to the plurality of extracted images are displayed in the second display region in a manner that enables identification of a correspondence relationship with the plurality of extracted images.

21. The medical support device according to claim 1, wherein
the extracted images are images extracted from the medical image using frames that make differences in size of the observation target regions visually distinguishable between the plurality of extracted images in a case in which the plurality of extracted images are compared.

22. The medical support device according to claim 21, wherein
the frames have a shape and a size that are common to the plurality of observation target regions.

23. The medical support device according to claim 1, wherein
the medical image is an endoscopic image obtained by imaging with an endoscope.

24. The medical support device according to claim 1, wherein
the observation target regions are lesions.

25. An endoscope system comprising:
the medical support device according to claim 1; and
an endoscope to be inserted into a body to acquire the medical image by imaging an inside of the body.

26. A medical support method comprising:
acquiring a characteristic of each of a plurality of observation target regions, the plurality of observation target regions being recognized by performing a recognition process on a medical image in which the plurality of observation target regions appear;
displaying the medical image in a first display region; and
displaying, in accordance with the characteristic, a plurality of extracted images in a second display region outside the first display region, the plurality of extracted images being images in which the plurality of observation target regions have been individually extracted from the medical image.

27. The medical support method according to claim 26, further comprising
using an endoscope that is inserted into a body and acquire the medical image by imaging an inside of the body.

28. A non-transitory computer-readable storage medium storing a program executable by a computer to execute a medical support process, the medical support process comprising:
acquiring a characteristic of each of a plurality of observation target regions, the plurality of observation target regions being recognized by performing a recognition process on a medical image in which the plurality of observation target regions appear;
displaying the medical image in a first display region; and
displaying, in accordance with the characteristic, a plurality of extracted images in a second display region outside the first display region, the plurality of extracted images being images in which the plurality of observation target regions have been individually extracted from the medical image.
